SEQUENCE LISTING (119 sequences)

SEQ ID NO: 1 - DNA, 4368 bp, synthetic construct
  misc_feature 1..4368: note = Empty pMiX4 vector backbone
  source 1..4368: mol_type = other DNA
aactgttgggaagggcgatcggtgcgggcctcttcgctattacgccagctggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagagttaacaccggtcgtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcacgtgtgagtttaaacgatctcatgctggagttcttcgcccacccccggcaataaaaagacagaataaaacgcacgggtgttgggtcgtttgttcgtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagcgagaccatgtggccacatggtctcgcttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttaggccgcactcgagatatcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgc

SEQ ID NO: 2 - DNA, 16 bp, synthetic construct
  misc_feature 1..16: note = miR-128-2 loop
  source 1..16: mol_type = other DNA
gagtgagtagcaggtc

SEQ ID NO: 3-6 - skipped (4 entries)

SEQ ID NO: 7 - DNA, 437 bp, synthetic construct
  misc_feature 1..437: note = pMiX4 transcript sequence
  source 1..437: mol_type = other DNA
gtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagttgtggtgtgatacgagaaactagagtgagtagcaggtctattctcgctgcacaccacgacttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttag

SEQ ID NO: 8 - DNA, 26 bp, synthetic construct
  misc_feature 1..26: note = Cloning sequence
  source 1..26: mol_type = other DNA
cgagaccatgtggccacatggtctcg

SEQ ID NO: 9 - DNA, 4967 bp, synthetic construct
  misc_feature 1..4967: note = Empty pMiX1 vector backbone
  source 1..4967: mol_type = other DNA
taacgccagggttttcccagtcacgacgttgtaaaacgacggccagtgacggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaagttaacaccggtgccgccatgggacacgggagcaagatggcgattccgggcaggcagtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagcgagaccatgtggccacatggtctcgcttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttaggaatattgttctgagaagccacagaaaaagggtggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcacgtgtgagtttaaacgatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttaggccgcactcgagatatctagacccagctttcttgtacaaagtggttgatctagagggcccggtacccagcttttgttccctttagtgagggttaattgcgcgcttggcgtaatcatggtcatagctgttttctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttggg

SEQ ID NO: 10 - DNA, 4898 bp, synthetic construct
  misc_feature 1..4898: note = Empty pMiX2 vector backbone
  source 1..4898: mol_type = other DNA
taacgccagggttttcccagtcacgacgttgtaaaacgacggccagtgacggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaagttaacaccggtcgtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagcgagaccatgtggccacatggtctcgcttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttaggggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcacgtgtgagtttaaacgatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttaggccgcactcgagatatctagacccagctttcttgtacaaagtggttgatctagagggcccggtacccagcttttgttccctttagtgagggttaattgcgcgcttggcgtaatcatggtcatagctgttttctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttggg

SEQ ID NO: 11 - DNA, 5006 bp, synthetic construct
  misc_feature 1..5006: note = Empty pMiX3 vector backbone
  source 1..5006: mol_type = other DNA
taacgccagggttttcccagtcacgacgttgtaaaacgacggccagtgacggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaagttaacaccggtcgtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcggaggcggaagcggacacgggagcaagatggcgattccgggcaggcagtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagcgagaccatgtggccacatggtctcgcttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttaggaatattgttctgagaagccacagaaaaaggcacgtgtacccctacgacgtccccgactacgcctgagtttaaacgatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttaggccgcactcgagatatctagacccagctttcttgtacaaagtggttgatctagagggcccggtacccagcttttgttccctttagtgagggttaattgcgcgcttggcgtaatcatggtcatagctgttttctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttggg

SEQ ID NO: 12 - DNA, 4489 bp, synthetic construct
  misc_feature 1..4489: note = Empty pMiX4-micro vector backbone
  source 1..4489: mol_type = other DNA
taacgccagggttttcccagtcacgacgttgtaaaacgacggccagtgacggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaagttaacaccggtcgtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcacgtgtgagtttaaacgatctcatgctggagttcttcgcccacccccggcaataaaaagacagaataaaacgcacgggtgttgggtcgtttgttcgtgaggaaataaaggaagttagcgagaccatgtggccacatggtctcgcttgcttcctaacccgcgcggccgcactcgagatatctagacccagctttcttgtacaaagtggttgatctagagggcccggtacccagcttttgttccctttagtgagggttaattgcgcgcttggcgtaatcatggtcatagctgttttctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttggg

SEQ ID NO: 13 - DNA, 4571 bp, synthetic construct
  misc_feature 1..4571: note = Empty pMiX4-mini vector backbone
  source 1..4571: mol_type = other DNA
taacgccagggttttcccagtcacgacgttgtaaaacgacggccagtgacggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaagttaacaccggtcgtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcacgtgtgagtttaaacgatctcatgctggagttcttcgcccacccccggcaataaaaagacagaataaaacgcacgggtgttgggtcgtttgttctacattttcccggatggaagcccgaagtttgagggagaaacttgtgaggaaataaaggaagttagcgagaccatgtggccacatggtctcgcttgcttcctaacccgcgcttgagtttctccccgcttggatgctctcaggggcagtgtggccgcactcgagatatctagacccagctttcttgtacaaagtggttgatctagagggcccggtacccagcttttgttccctttagtgagggttaattgcgcgcttggcgtaatcatggtcatagctgttttctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttggg

SEQ ID NO: 14 - DNA, 436 bp, synthetic construct
  misc_feature 1..436: note = pMiX4.1 transcript sequence
  source 1..436: mol_type = other DNA
ggtcgaatcagttagtaacccgtcccggttgaacagagagggggtaggttgaagttgtttaagcgtcggtgtgaaagacccactaagatacgattgtcagacacttattacttgaggctgagcgagttcacggcacatagtacaggagaacatcagcaacggcgtacattttcccggatggaagcccgaagtttgagggagaaacttgtgaggaaataaaggaagttagttgtggtgtgatacgagaaactagagtgagtagcaggtctattctcgctgcacaccacgacttgcttcctaacccgcgcttgagtttctccccgcttggatgctctcaggggcagtgtctgtaactacaatcatcccagttccccctaccactaatattagaatagctctcataagtcgctagatcagactctgtgactgctcaata

SEQ ID NO: 15 - DNA, 436 bp, synthetic construct
  misc_feature 1..436: note = pMiX4.2 transcript sequence
  source 1..436: mol_type = other DNA
ggtcgaatcagttagtaacccgtcccggttgaacagagagggggtaggttgaagttgtttaagcgtcggtgtgaaagacccactaagatacgattgtcagacacttattacttgaggctgagcgagttcacggcacatagtacaggagaacatcagcaacggcgcaaattagttactgagttaacagtgactcgccagcaataaggggtgaggaaataaaggaagttagttgtggtgtgatacgagaaactagagtgagtagcaggtctattctcgctgcacaccacgacttgcttcctaacccgcgcgtgataacgcaaccagtcgttcaaactcgcacacccttgctgtaactacaatcatcccagttccccctaccactaatattagaatagctctcataagtcgctagatcagactctgtgactgctcaata

SEQ ID NO: 16 - DNA, 436 bp, synthetic construct
  misc_feature 1..436: note = pMiX4.3 transcript sequence
  source 1..436: mol_type = other DNA
ggtcgaatcagttagtaacccgtcccggttgaacagagagggggtaggttgaagttgtttaagcgtcggtgtgaaagacccactaagatacgattgtcagacacttattacttgaggctgagcgagttcacggcacatagtacaggagaacatcagcaacggcgcaaattagttactgagttaacagtgactcgccagcaataaggggtaaaggagcttaggaagttagttgtggtgtgatacgagaaactagagtgagtagcaggtctattctcgctgcacaccacgacttgcttcctgcactaatcgtgataacgcaaccagtcgttcaaactcgcacacccttgctgtaactacaatcatcccagttccccctaccactaatattagaatagctctcataagtcgctagatcagactctgtgactgctcaata

SEQ ID NO: 17 - DNA, 436 bp, synthetic construct
  misc_feature 1..436: note = pMiX4.4 transcript sequence
  source 1..436: mol_type = other DNA
ggtcgaatcagttagtaacccgtcccggttgaacagagagggggtaggttgaagttgtttaagcgtcggtgtgaaagacccactaagatacgattgtcagacacttattacttgaggctgagcgagttcacggcacatagtacaggagaacatcagcaacggcgcaaattagttactgagttaacagtgactcgccagcaataaggggtaaaggagcttagctcctgatttgtggtgtgatacgagaaactagagtgagtagcaggtctattctcgctgcacaccacgagagatacaacgcactaatcgtgataacgcaaccagtcgttcaaactcgcacacccttgctgtaactacaatcatcccagttccccctaccactaatattagaatagctctcataagtcgctagatcagactctgtgactgctcaata

SEQ ID NO: 18 - DNA, 219 bp, synthetic construct
  misc_feature 1..219: note = miR 423 5' intronic sequence
  source 1..219: mol_type = other DNA
gtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataa

SEQ ID NO: 19 - DNA, 10 bp, synthetic construct
  misc_feature 1..10: note = miR 423 5' stem
  source 1..10: mol_type = other DNA
aggaagttag

SEQ ID NO: 20 - DNA, 10 bp, synthetic construct
  misc_feature 1..10: note = miR 423 3' stem
  source 1..10: mol_type = other DNA
cttgcttcct

SEQ ID NO: 21 - DNA, 138 bp, synthetic construct
  misc_feature 1..138: note = miR 423 3' intronic sequence
  source 1..138: mol_type = other DNA
aacccgcgcttgagtttctccccgcttggatgctctcaggggcagtgtgaagagagacagttcctggcttccttagagggccttgttctccttggcagaagctcaagtcctgattcccgcccttccttctcgttttag

SEQ ID NO: 22-41 - skipped (20 entries)

SEQ ID NO: 42 - DNA, 33 bp, synthetic construct
  misc_feature 1..33: note = Full SR binding sequence of pMix 4.1 5'
  source 1..33: mol_type = other DNA
ttcccggatggaagcccgaagtttgagggagaa

SEQ ID NO: 43 - DNA, 25 bp, synthetic construct
  misc_feature 1..25: note = Full SR binding sequence of pMix 4.1 3'
  source 1..25: mol_type = other DNA
ttctccccgcttggatgctctcagg

SEQ ID NO: 44-45 - skipped (2 entries)

SEQ ID NO: 46 - DNA, 4673 bp, synthetic construct
  misc_feature 1..4673: note = Empty pMiX6 vector backbone
  source 1..4673: mol_type = other DNA
taacgccagggttttcccagtcacgacgttgtaaaacgacggccagtgacggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaagttaacaccggtcgtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcacgtgtgagtttaaactaagcacttcgtggccgtcgatcgtttaaagggaggtagtgagtcgaccagtggatcctggaggcttgctgaaggctgtatgctcgagaccatgtggccaatgggtctcccaggacacaaggcctgttactagcactcacatggaacaaatggcccagatctggccgcactcgaaaacgatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttaggccgcactcgagatatctagacccagctttcttgtacaaagtggttgatctagagggcccggtacccagcttttgttccctttagtgagggttaattgcgcgcttggcgtaatcatggtcatagctgttttctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttggg

SEQ ID NO: 47 - DNA, 4872 bp, synthetic construct
  misc_feature 1..4872: note = Empty pMiX6.4 vector backbone
  source 1..4872: mol_type = other DNA
taacgccagggttttcccagtcacgacgttgtaaaacgacggccagtgacggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaagttaacaccggtcgtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcacgtgtgagtttaaacgatctcatgctggagttcttcgcccacccccggcaataaaaagacagaataaaacgcacgggtgttgggtcgtttgttcgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgtttaaactaagcacttcgtggccgtcgatcgtttaaagggaggtagtgagtcgaccagtggatcctggaggcttgctgaaggctgtatgctcgagaccatgtggccaatgggtctcccaggacacaaggcctgttactagcactcacatggaacaaatggcccagatctggccgcactcgaaaacgatctcatgctggagttcttcgcccaccccattaatgaatcggccaacgcgcggggagaggcggtttgcgtattgggcgctcttccgcttcctcgctcactgactcgctgcgctggccgcactcgagatatctagacccagctttcttgtacaaagtggttgatctagagggcccggtacccagcttttgttccctttagtgagggttaattgcgcgcttggcgtaatcatggtcatagctgttttctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttggg

SEQ ID NO: 48 - DNA, 4916 bp, synthetic construct
  misc_feature 1..4916: note = Empty pMiX7 vector backbone
  source 1..4916: mol_type = other DNA
ttcccagtcacgacgttgtaaaacgacggccagtgacggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaagttaacaccggtcgtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgggccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcacgtgtgagtttaaacgatctcatgcttgtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagcgagaccatgtggccacatggtctcgcttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttaggccgcactcgagatatctgagttcttcgcccaccccaacttgtttattgcagcttataatggttacaaataaagcaatagcatcacaaatttcacaaataaagcatttttttcactgcattctagttgtggtttgtccaaactcatcaatgtatcttaggccgcactcgagatatctagacccagctttcttgtacaaagtggttgatctagagggcccggtacccagcttttgttccctttagtgagggttaattgcgcgcttggcgtaatcatggtcatagctgttttctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggtt

SEQ ID NO: 49 - DNA, 81 bp, synthetic construct
  misc_feature 1..81: note = miR 423 hairpin seq
  source 1..81: mol_type = other DNA
aggaagttaggctgaggggcagagagcgagacttttctattttccaaaagctcggtctgaggcccctcagtcttgcttcct

SEQ ID NO: 50 - DNA, 77 bp, synthetic construct
  misc_feature 1..77: note = miR-26b hairpin seq
  source 1..77: mol_type = other DNA
ccgggacccagttcaagtaattcaggataggttgtgtgctgtccagcctgttctccattacttggctcggggaccgg

SEQ ID NO: 51 - DNA, 85 bp, synthetic construct
  misc_feature 1..85: note = miR-126 hairpin seq
  source 1..85: mol_type = other DNA
cgctggcgacgggacattattacttttggtacgcgctgtgacacttcaaactcgtaccgtgagtaataatgcgccgtccacggca

SEQ ID NO: 52 - DNA, 82 bp, synthetic construct
  misc_feature 1..82: note = miR -106b hairpin seq
  source 1..82: mol_type = other DNA
cctgccggggctaaagtgctgacagtgcagatagtggtcctctccgtgctaccgcactgtgggtacttgctgctccagcagg

SEQ ID NO: 53 - DNA, 80 bp, synthetic construct
  misc_feature 1..80: note = miR -93 hairpin seq
  source 1..80: mol_type = other DNA
ctgggggctccaaagtgctgttcgtgcaggtagtgtgattacccaacctactgctgagctagcacttcccgagcccccgg

SEQ ID NO: 54 - DNA, 84 bp, synthetic construct
  misc_feature 1..84: note = miR -25 hairpin seq
  source 1..84: mol_type = other DNA
ggccagtgttgagaggcggagacttgggcaattgctggacgctgccctgggcattgcacttgtctcggtctgacagtgccggcc

SEQ ID NO: 55-58 - skipped (4 entries)

SEQ ID NO: 59 - DNA, 12 bp, synthetic construct
  misc_feature 1..12: note = miR 126 STEM 5'
  source 1..12: mol_type = other DNA
cgctggcgacgg

SEQ ID NO: 60-61 - skipped (2 entries)

SEQ ID NO: 62 - DNA, 11 bp, synthetic construct
  misc_feature 1..11: note = miR 25 STEM 5'
  source 1..11: mol_type = other DNA
ggccagtgttg

SEQ ID NO: 63 - DNA, 10 bp, synthetic construct
  misc_feature 1..10: note = miR 26b STEM 3'
  source 1..10: mol_type = other DNA
cggggaccgg

SEQ ID NO: 64 - DNA, 12 bp, synthetic construct
  misc_feature 1..12: note = miR 126 STEM 3'
  source 1..12: mol_type = other DNA
ccgtccacggca

SEQ ID NO: 65-66 - skipped (2 entries)

SEQ ID NO: 67 - DNA, 11 bp, synthetic construct
  misc_feature 1..11: note = miR 25 STEM 3'
  source 1..11: mol_type = other DNA
cagtgccggcc

SEQ ID NO: 68 - DNA, 18 bp, synthetic construct
  misc_feature 1..18: note = pcDNA6.2 passenger of mir-Fluc1
  source 1..18: mol_type = other DNA
cgtggtgcagcgagaata

SEQ ID NO: 69 - DNA, 78 bp, synthetic construct
  misc_feature 1..78: note = miR-128-2 hairpin
  source 1..78: mol_type = other DNA
gcagtgggaaggggggccgatacactgtacgagagtgagtagcaggtctcacagtgaaccggtctctttccctactgt

SEQ ID NO: 70 - DNA, 13 bp, synthetic construct
  misc_feature 1..13: note = miR 423 loop
  source 1..13: mol_type = other DNA
tctattttccaaa

SEQ ID NO: 71 - DNA, 14 bp, synthetic construct
  misc_feature 1..14: note = miR-26b loop
  source 1..14: mol_type = other DNA
tgtgtgctgtccag

SEQ ID NO: 72 - DNA, 19 bp, synthetic construct
  misc_feature 1..19: note = miR 126 loop
  source 1..19: mol_type = other DNA
agtggtcctctccgtgcta

SEQ ID NO: 73 - DNA, 19 bp, synthetic construct
  misc_feature 1..19: note = miR 106b loop
  source 1..19: mol_type = other DNA
agtggtcctctccgtgcta

SEQ ID NO: 74 - DNA, 16 bp, synthetic construct
  misc_feature 1..16: note = miR 93 loop
  source 1..16: mol_type = other DNA
tgtgattacccaacct

SEQ ID NO: 75 - DNA, 17 bp, synthetic construct
  misc_feature 1..17: note = miR 25 loop
  source 1..17: mol_type = other DNA
ctggacgctgccctggg

SEQ ID NO: 76-83 - skipped (8 entries)

SEQ ID NO: 84 - DNA, 240 bp, synthetic construct
  misc_feature 1..240: note = miR 26b 5' intronic sequence
  source 1..240: mol_type = other DNA
gtgagggccaggaagaggcagtggtgggcttggcatctgcctccagaccctaggctcttcccaccaatccggagcgcctcggatgggaattggatacatgtggaatgtcagaggcccagagagggtgtgagacttgtcccaaagtcacacagaacctcaagggcttgtgctgactccaagcctgcagagtgggctcctcctctaggctcccccgtgctgtgctccctcgccccaccctgc

SEQ ID NO: 85 - DNA, 271 bp, synthetic construct
  misc_feature 1..271: note = miR 126 5' intronic sequence
  source 1..271: mol_type = other DNA
gtaaacagccctggctgtgcctggcctggggaggcgggcaggcagtggacattgccgtgtggctgttaggcatggtggggggcactggaatctgggcggaaggcggtggggactccctctccagggagggaggatggggagggaggataggtgggttcccgagaactgggggcaggttgcccggagcctcatatcagccaagaaggcagaagtgccccgtcccggggtcctgtctgcatccagcgcagcattctggaagacgccacgcctc

SEQ ID NO: 86 - DNA, 98 bp, synthetic construct
  misc_feature 1..98: note = miR 106b 5' intronic sequence
  source 1..98: mol_type = other DNA
gtaagtgcccaaattgctggagggccatctgttttgacccttaaaggggtagctccttaccgtgctctcattgccgcctccccacctcccgctccagc

SEQ ID NO: 87 - DNA, 145 bp, synthetic construct
  misc_feature 1..145: note = miR 93 5' intronic sequence
  source 1..145: mol_type = other DNA
gcacgcacagcgtccgtggagggaaaggccttttccccacttcttaaccttcactgagagggtggttggggtctgtttcactccatgtgtcctagatcctgtgctacagaccttcctttctgtcctcccgtcttggacctcagtc

SEQ ID NO: 88 - DNA, 124 bp, synthetic construct
  misc_feature 1..124: note = miR 25 5' intronic sequence
  source 1..124: mol_type = other DNA
gacacgttctctctgccaattgtcctcttggctgagctccccaagctccatctgtcatgctggggagcccagtggcgttcaaaagggtctggactccctcacaggacagctgaactccgggact

SEQ ID NO: 89 - DNA, 312 bp, synthetic construct
  misc_feature 1..312: note = miR 26b 3' intronic sequence
  source 1..312: mol_type = other DNA
tgccctgcagccttggggtgagggggctgcccctggattcctgcactaggctgaggttgaggcaggggaagggattgggaattagggacctcgtgaggtaggactggccagtggagtggaagttttgatcgttttctggcggggggtgggtacagtttccccagcagtggtcagggtagctggccaagcggagcctgcgggcccagtctccttcctgtgcgcctctgcctccctggcccatgccctgccagccctcggccacccccacactgccccactggcccgcagccccctcactggcccgccccccag

SEQ ID NO: 90 - DNA, 263 bp, synthetic construct
  misc_feature 1..263: note = miR 126 3' intronic sequence
  source 1..263: mol_type = other DNA
ccgcatcgaaaacgccgctgagtcctcagccttgacctccctcagcgtggccgggaccctgagcctctgcgcagagccacccgccccgacgtacttaggcggcatagccctgagtcctctggccagcgccaggcaggcagcgggggcggcagaggcctgggcctgagtcttctggctctgcctctccctggggacaggagggagcctgggggtgtgggtggggagccggccggccgtgacccagcgcctggctctgcccgcag

SEQ ID NO: 91 - DNA, 145 bp, synthetic construct; identical to SEQ ID NO: 87
  misc_feature 1..145: note = miR 106b 3' intronic sequence
  source 1..145: mol_type = other DNA

SEQ ID NO: 92 - DNA, 124 bp, synthetic construct; identical to SEQ ID NO: 88
  misc_feature 1..124: note = miR 93 3' intronic sequence
  source 1..124: mol_type = other DNA

SEQ ID NO: 93 - DNA, 158 bp, synthetic construct
  misc_feature 1..158: note = miR 25 3' intronic sequence
  source 1..158: mol_type = other DNA
caacactgcggatgctggggggaggggggattccactcctgttttgtgagtaggcgacccatgggctgcccagccttaaagccagaacaagggtgtcccctgacctcgttccactgccctcctcccgttcccatctttcccccctaccttccccttag

SEQ ID NO: 94 - DNA, 21 bp, synthetic construct
  misc_feature 1..21: note = Fluc1 amiR
  source 1..21: mol_type = other DNA
tattctcgctgcacaccacga

SEQ ID NO: 95 - DNA, 21 bp, synthetic construct
  misc_feature 1..21: note = 128 amiR
  source 1..21: mol_type = other DNA
tcacagtgaaccggtctcttt

SEQ ID NO: 96 - DNA, 17 bp, synthetic construct
  misc_feature 1..17: note = pMiX1 primer 1
  source 1..17: mol_type = other DNA
agtcccaagctggctag

SEQ ID NO: 97 - DNA, 16 bp, synthetic construct
  misc_feature 1..16: note = pMiX1 primer 2
  source 1..16: mol_type = other DNA
ccggacacgctgaact

SEQ ID NO: 98 - DNA, 20 bp, synthetic construct
  misc_feature 1..20: note = pMiX2 primer 1
  source 1..20: mol_type = other DNA
cagcacgacttcttcaagtc

SEQ ID NO: 99 - DNA, 18 bp, synthetic construct
  misc_feature 1..18: note = pMiX2 primer 2
  source 1..18: mol_type = other DNA
tgtactccagcttgtgcc

SEQ ID NO: 100 - DNA, 18 bp, synthetic construct
  misc_feature 1..18: note = pMiX3 primer 1
  source 1..18: mol_type = other DNA
cgatcacatggtcctgct

SEQ ID NO: 101 - DNA, 22 bp, synthetic construct
  misc_feature 1..22: note = pMiX3 primer 2
  source 1..22: mol_type = other DNA
cacacagaaaacagctatgacc

SEQ ID NO: 102 - DNA, 20 bp, synthetic construct
  misc_feature 1..20: note = Firefly primer 1
  source 1..20: mol_type = other DNA
gtggtgtgcagcgagaatag

SEQ ID NO: 103 - DNA, 22 bp, synthetic construct
  misc_feature 1..22: note = Firefly primer 2
  source 1..22: mol_type = other DNA
cgctcgttgtagatgtcgttag

SEQ ID NO: 104 - DNA, 20 bp, synthetic construct
  misc_feature 1..20: note = Renilla primer 1
  source 1..20: mol_type = other DNA
gagaagggcgaggttagacg

SEQ ID NO: 105 - DNA, 20 bp, synthetic construct
  misc_feature 1..20: note = Renilla primer 2
  source 1..20: mol_type = other DNA
tggaaaagaacccagggtcg

SEQ ID NO: 106 - DNA, 22 bp, synthetic construct
  misc_feature 1..22: note = GAPDH primer 1
  source 1..22: mol_type = other DNA
gtctcctctgacttcaacagcg

SEQ ID NO: 107 - DNA, 22 bp, synthetic construct
  misc_feature 1..22: note = GAPDH primer 2
  source 1..22: mol_type = other DNA
accaccctgttgctgtagccaa

SEQ ID NO: 108 - DNA, 22 bp, synthetic construct
  misc_feature 1..22: note = Drosha primer 1
  source 1..22: mol_type = other DNA
taggctgtgggaaaggaccaag

SEQ ID NO: 109 - DNA, 22 bp, synthetic construct
  misc_feature 1..22: note = Drosha primer 2
  source 1..22: mol_type = other DNA
gttcgatgaaccgcttctgatg

SEQ ID NO: 110 - DNA, 18 bp, synthetic construct
  misc_feature 1..18: note = miR-128 primer
  source 1..18: mol_type = other DNA
tcacagtgaaccggtctc

SEQ ID NO: 111 - DNA, 17 bp, synthetic construct
  misc_feature 1..17: note = pMiX passenger of mir-128
  source 1..17: mol_type = other DNA
aggggggccgatacact

SEQ ID NO: 112 - DNA, 18 bp, synthetic construct
  misc_feature 1..18: note = pcDNA6.2 passenger of mir-128
  source 1..18: mol_type = other DNA
aaagagacgttcactgtg

SEQ ID NO: 113 - DNA, 19 bp, synthetic construct
  misc_feature 1..19: note = mir-Fluc1
  source 1..19: mol_type = other DNA
tattctcgctgcacaccac

SEQ ID NO: 114 - DNA, 21 bp, synthetic construct
  misc_feature 1..21: note = pMiX passenger of mir-Fluc1
  source 1..21: mol_type = other DNA
ttgtggtgtgatacgagaaac

SEQ ID NO: 115 - DNA, 80 bp, synthetic construct
  misc_feature 1..80: note = miR 423-128 hybrid hairpin
  source 1..80: mol_type = other DNA
aggaagttagcttggggtacatagcgtagacgtgagtgagtagcaggtcacctacgccgagtacttcgagcttgcttcct

SEQ ID NO: 116 - DNA, 4402 bp, synthetic construct
  misc_feature 1..4402: note = Empty pMiX4B (scrambled sequence) vector backbone
  misc_difference 1775..1784: note = n is a, c, g, or t
  misc_difference 1788..1793: note = n is a, c, g, or t
  misc_difference 1796..1797: note = n is a, c, g, or t
  misc_difference 1814..1834: note = n is a, c, g, or t
  source 1..4402: mol_type = other DNA
aactgttgggaagggcgatcggtgcgggcctcttcgctattacgccagctggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagagtggtgattgtatctgaacggaattactatactaacgataaggttatagtgcccggcggagattattgcaataaggattcgagatatagtaactgtttgcgttgataggaagtatcgtatcttcgaagaggagagggtttttggactccaccgagtggttggcacacactgagttagcacaaattgcccagaataatattataaggactcaaactaagggtttattccataaccccgaattttatatcgcgccgaagtgcataccccgactattgcctttcgggccttacaatccctagcaattaacgaaccaagatcttcaatttacttatataaggggttgtgtaggtcgaatcagttagtaacccgtcccggttgaacagagagggggtaggttgaagttgtttaagcgtcggtgtgaaagacccactaagatacgattgtcagacacttattacttgaggctgagcgagttcacggcacatagtacaggagaacatcagcaacggcgcaaattagttactgagttaacagtgactcgccagcaataaggggtaaaggagcttagctcctgattgcgattagtgcatcaaacgttgatttggggatattaatctagtagacaactcgaaaatttgttacaaatcagaaagctgaaagtagtcggtacctattagatcttcaacggcacaagttgcgggccgtgtgcttcaaacgcatcagagcgtttggtatcacgtgtgagtttaaacgatctcatgctggagttcttcgcccacccccggcaataaaaagacagaataaaacgcacgggtgttgggtcgtttgttcgtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagnnnnnnnnnnatannnnnnacnngagtgagtagcaggtcnnnnnnnnnnnnnnnnnnnnncttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttaggccgcactcgagatatcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgc

SEQ ID NO: 117 - DNA, 4480 bp, synthetic construct
  misc_feature 1..4480: note = Empty pMiX4C (NeoR/KanR) vector backbone
  misc_difference 1839..1848: note = n is a, c, g, or t
  misc_difference 1852..1857: note = n is a, c, g, or t
  misc_difference 1860..1861: note = n is a, c, g, or t
  misc_difference 1878..1898: note = n is a, c, g, or t
  source 1..4480: mol_type = other DNA
gcgatcggtgcgggcctcttcgctattacgccagctggccgctctagaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgccaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagagttaacaccggtcgtctgatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacacgtgtgagtttaaacgatctcatgctggagttcttcgcccacccccggcaataaaaagacagaataaaacgcacgggtgttgggtcgtttgttcgtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagnnnnnnnnnnatannnnnnacnngagtgagtagcaggtcnnnnnnnnnnnnnnnnnnnnncttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttaggccgcactcgagatatcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcatcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagg

SEQ ID NO: 118 - DNA, 437 bp, synthetic construct
  misc_feature 1..437: note = 423 intron with hybrid hairpin
  source 1..437: mol_type = other DNA
gtgagtgatccgggagttagggtcaggctgggggatgagaaactacggcgactgtatctttggccgaggagttttaaatgcgctggaagtgaagggactgtgtcgtcaagggtagagacgggaaaaaacgagaagtcctgaggaacatagattcccctcccccgtacattttcccggatggaagcccgaagtttgagggagaaacttgtgaggaaataaaggaagttagcttggggtacatagcgtagacgtgagtgagtagcaggtcacctacgccgagtacttcgagcttgcttcctaacccgcgcttgagtttctccccgcttggatgctctcaggggcagtgtgaagagagacagttcctggcttccttagagggccttgttctccttggcagaagctcaagtcctgattcccgcccttccttctcgttttag

SEQ ID NO: 119 - DNA, 437 bp, synthetic construct; identical to SEQ ID NO: 118
  source 1..437: mol_type = other DNA

(19) United States
(12) Patent Application Publication
(10) Pub. No.: US 2025/0270551 A1
(43) Pub. Date: Aug. 28, 2025

(54) MICRORNA SYSTEM

(71) Applicant: King's College London, London (GB)

(72) Inventors: Christopher SHAW, London (GB); Youn Bok LEE, London (GB); Romain JOUBERT, London (GB); Do Young LEE, London (GB)

(21) Appl. No.: 18/687,987

(22) PCT Filed: Sep. 1, 2022
(86) PCT No.: PCT/EP2022/074398

§ 371 Date: Feb. 29, 2024

(30) Foreign Application Priority Data
Sep. 1, 2021 (GB) 2112471.4

(51) Int. Cl. C12N 15/113

(52) U.S. Cl. CPC C12N 15/113; C12N 2310/141; C12N 2310/531

(57) ABSTRACT

Described herein are microRNA expression systems, and/or microRNA cloning systems. Described herein are also methods of expressing microRNAs or guide sequences against target mRNA, methods of producing said microRNA cloning systems and methods of knocking down target polynucleotides, such as mRNA.

### SEQUENCE LISTING

The instant application contains a Sequence Listing which has been submitted electronically in XML format and is hereby incorporated by reference in its entirety. Said XML copy, created on Sep. 12, 2024, is named “51585-008002_Sequence_Listing_9_12_24” and is 166,674 bytes in size.

### FIELD OF THE INVENTION

The present invention relates to microRNA expression systems, and/or microRNA cloning systems. The present invention also relates to methods of expressing microRNAs or guide sequences against target mRNA, methods of producing said microRNA cloning systems and methods of knocking down target polynucleotides, such as mRNA.

### BACKGROUND OF THE INVENTION

Plants and animals generate short RNA sequences that are complementary to, and bind to, specific mRNA transcripts to form double stranded RNA and thereby inhibit translation or facilitate degradation. This discovery led to the exploitation of RNA interference (RNAi) to knock down genes as a powerful tool to explore a multitude of biology systems (Fire et al, 1998. Nature, 1998. 391(6669): p. 806-11) and resulted in the 2006 Nobel Prize for Physiology and Medicine being awarded to Craig Mello and Andrew Fire.

RNAi has also spawned a range of therapeutic strategies to knock down mutant genes that were pathogenic or in disease-critical pathways or affect transcript splicing to enhance normal protein translation. The first therapies to be brought to clinical trial are antisense oligonucleotides (ASO), short fragments of modified DNA which bind to target mRNA leading to their degradation by RNAse H (Bennet and Swayze, 2010. Annual Review of Pharmacology and Toxicology. 50: 259-93). One of the most successful ASO clinical trials targeted superoxide dismutase 1 (SOD1) gene mutations in which cause amyotrophic lateral sclerosis (ALS) (unpublished link). ASO targeting neurological disorders are given intrathecally via a lumbar puncture at monthly intervals which is expensive and invasive. An attractive alternative is to deliver genes encoding Short hairpin RNA (shRNA) and micro RNA (miRNA). These are derived from much longer RNA sequences that are processed initially in the nucleus and subsequently in the cytoplasm where they bind to, and inhibit, target mRNA (Han et al, 2006. Cell 125, 887-901) (FIG. 1).

After transcription, miRNAs transcripts undergo 5′ capping and 3′ polyadenylation to generate primary miRNA (pri-miRNA) which are initially processed by the DGCR8/Drosha complex to a shorter precursor miRNA (pre-miRNA). Pre-miRNA from more stable hairpin structures and are transported by Exportin-5 from the nucleus to the cytoplasm, where they undergo further editing by the Dicer complex. This cleavage generates a 22-nucleotide duplex miRNA, which includes a guide RNA strand that can enter the RNA-induced silencing complex (RISC) and bind to its target RNA, while the passenger strand is degraded. If the guide strand is perfectly complementary to the target sequence the catalytic component of RISC, argonaut 2 (AGO2) will degrade the target RNA, but if complementarity is restricted to a short ‘seed sequence’ it will only suppress translation.

Engineered RNAi offer potentially powerful gene therapies and their design exploits the physiological processing of endogenous miRNA. shRNAs are transcribed by strong Pol III promoters (such as H1 and U6) and are generated as preformed hairpins. They do not need Drosha cleavage and follow the same processing pathway as miRNA. High levels of shRNA by Pol III promoters however can overload RNAi processing pathways leading to toxicity (McBride et al, 2008. PNAS, 105(15): 5868-5873). miRNAs are transcribed by Pol II promoters at lower levels than Pol III promoters thereby reducing the risk of toxicity due to saturation of RNA processing machinery. They can also be placed under cell-type specific promoters further reducing the risk of toxicity. For this reason, we have chosen to develop miRNA over shRNA for our long-term gene silencing therapeutic strategy.

In order to reach target cells, shRNA and miRNA must be delivered by carriers, such as viral and non-viral vectors. While each have advantages, viral vectors likely have greater potential for clinical use. Within the realm of viral vectors, Lentivirus integrate into the genomic sequence while Adeno-associated virus (AAV) remain episomal (non-integrating). Lentiviral vectors are able to transduce dividing and non-dividing cells, which can be of benefit when targeting neurons (Deglon et al., 2000. Hum. Gene Ther., 11: 179-190), however, insertional mutagenesis and inflammation causing death has been reported in patients treated with lentiviral viral vectors limiting their utility (Persons and Baum, 2011. Mol. Ther., 19: 229-231).

AAV is much less immunogenic and rarely integrates into the genome. Most AAV-delivered therapies aim to treat disorders caused by loss of function mutations by supplementing defective gene expression. AAV can also be used to deliver genes encoding miRNA that knock down mutant genes that have a toxic gain of function or are pivotal to disease pathogenesis. Many late-onset neurodegenerative diseases are due to the accumulation of mutant and misfolded proteins making them ideal targets for miRNA therapies and slowing the disease progression (Maciotta et al., 2013. Front. Cell. Neurosci., 7: 265).

AAV5-mediated knock-down of the HTT gene was achieved in a rat model of Huntington's disease using shRNA sequence embedded within a miRNA structure (Miniarikova et al., 2017. Gene Therapy, 24(10): 630-639). AAV5-miHTT reduce the accumulation of toxic HTT protein and improved motor coordination. AAV9 delivery of shRNA and artificial miRNA targeting SOD1 lead to a delay in disease progression and increased survival in a transgenic mouse model of ALS (Foust et al., 2013. Mol Ther, 21(12):2148-2159, Stoica et al, 2016. Ann Neurol. 79(4): 687-700). Recently, AAV-miR-SOD1 was injected into the tongue and pleural space targeting the respiratory motor neurons of transgenic mice, which prolonged survival by ˜50 days (Keeler et al, 2020. Mol. Ther. Methods Clin. Dev. 17: 246-257). Collectively, these studies show that engineered miRNA can arrest devastating neurodegenerative diseases such as ALS.

The most commonly used, and commercially available, miRNA cloning system is pCDNA 6.2-GW/EMGFP-miR (BLOCK-iTTM, Thermofisher). These constructs express artificial miRNAs engineered to have 100% homology to target sequence and result in target mRNA cleavage. However, this system has several disadvantages. Firstly, the pcDNA6.2 miRNA cloning system is not particularly efficient at targeting 5′ untranslated region (UTR) and the coding sequence (CDS) of mRNA, achieving, on average, respectively 38% and 30% knock down in our experiments. pcDNA6.2 is therefore only effective at targeting the 3′UTR of target mRNA at about 65% knockdown. Furthermore, pcDNA6.2 results in 15-25% of total miRNA expression being of the passenger strand, rather than the guide strand, which is thought to lead to increased off-target effects and reduced levels of guide strands in the RISC. pcDNA6.2 also results in slower knockdown of target mRNA, such that the time required for maximum knockdown is around 24 hours.

Accordingly, there is a need for an miRNA cloning system that overcomes one or more of these issues. In particular, there is a need for an miRNA cloning system which allows efficient targeting of the 5′ and 3′ UTR, along with the CDS. In addition, there is a need for an miRNA cloning system which is more efficient at knocking down target mRNA than the commercial cloning system. In addition, there is a need for an miRNA cloning system that is able to load a higher ratio of guide miRNA strands to passenger miRNA strands into the RISC than the commercial cloning system, thereby providing a miRNA cloning system which also results in reduced off target effects compared to the commercial cloning system. Finally, there is a need for an miRNA cloning system which results in faster processing of miRNA and quicker knockdown of target mRNA.

### SUMMARY OF THE INVENTION

Accordingly, in one aspect the present invention provides a hybrid microRNA or precursor thereof comprising:
a stem sequence from a first intronic microRNA;
a loop sequence from a second microRNA, wherein the second microRNA is different to the first intronic microRNA; and
a guide sequence that binds specifically to a target mRNA;
wherein the stem, loop and guide sequences together form a hybrid hairpin that is processed to form a mature microRNA comprising the guide sequence, and the mature microRNA directs degradation of and/or inhibits translation of said target mRNA.

In one embodiment, the loop sequence is derived from miR-128-2, preferably wherein the loop sequence comprises, consists or consists essentially of SEQ ID NO: 2 or a sequence having at least 85%, 90%, 95% or 99% sequence identity to SEQ ID NO: 2.

In one embodiment, the stem sequence is derived from miR-423, miR-26b, miR-126, miR-106b, miR-93, miR-25, preferably wherein the stem sequence comprises, consists or consists essentially of any one of SEQ ID NOs: 19-20 and 58-67 or a sequence having at least 85%, 90%, 95% or 99% sequence identity to any one of SEQ ID NOs: 19-20 and 58-67.

In one embodiment, the stem sequence is derived from miR-423, preferably wherein the stem sequence comprises, consists or consists essentially of SEQ ID NOs: 19-20, or a sequence having at least 85%, 90%, 95% or 99% sequence identity to SEQ ID NOs: 19-20.

In one embodiment the hybrid microRNA or precursor thereof is a pre-microRNA or pri-microRNA.

In one embodiment, the hybrid microRNA or precursor thereof further comprises an intronic sequence 5′ to the stem and loop sequences, preferably wherein the 5′ intronic sequence is derived from miR-423, more preferably wherein the 5′ intronic sequence comprises, consists or consists essentially of SEQ ID NO: 18 or any one of SEQ ID Nos: 84-88, or a sequence having at least 85%, 90%, 95% or 99% sequence identity to SEQ ID NO: 18 or any one of SEQ ID Nos: 84-88.

In one embodiment, the 5′ intronic sequence comprises at least 30, 50, 100, 150 or 200 nucleotide residues, more preferably wherein the 5′ intronic sequence comprises 50 to 250 or 100 to 250 nucleotide residues, more preferably wherein the 5′ intronic sequence comprises at least 50, 100, 150 or 200 nucleotide residues of, or the full length of SEQ ID NO: 18 or any one of SEQ ID Nos: 84-88.

In one embodiment, the hybrid microRNA or precursor further comprises an intronic sequence 3′ to the stem and loop sequences, preferably wherein the 3′ intronic sequence is derived from miR-423, more preferably wherein the 3′ intronic sequence comprises, consists, or consists essentially of SEQ ID NO: 21 or any one of SEQ ID Nos: 89-93, or a sequence having at least 85%, 90%, 95% or 99% sequence identity to SEQ ID NO: 21 or any one of SEQ ID Nos: 89-93.

In one embodiment, the hybrid microRNA or precursor thereof comprises one or more serine/arginine rich protein (SR protein) binding sites.

In one embodiment, the one or more SR protein binding sites are 5′ to the stem and loop sequences and comprises or consists of: SRSF5-(SEQ ID NOs: 22 or 23), SRSF1-(SEQ ID NOs: 24 or 25), SRSF1-HMG (SEQ ID NO: 26), SRSF-(SEQ ID NO: 27), SRSF1-(SEQ ID NO: 36), SRSF6-(SEQ D NO: 37) or SRSF5-(SEQ ID NO: 38).

In one embodiment, the one or more SR binding sites are 3′ and comprises or consists of: SRSF6-(SEQ ID NO: 28), SRSF5-(SEQ ID NOs: 29 or 30), SRSF1-HMG-(SEQ ID NOs: 31 to 33), SRSF1-(SEQ ID NO: 34), SRSF2-(SEQ ID NO: 35), SRSF6-(SEQ ID NO: 39) or SRSF2-(SEQ ID NOs: 40 and 41).

In one embodiment, the guide sequence is between 12-30 nucleotides in length.

In one embodiment, the target mRNA is an mRNA from (i) a gene for which a mutation results in a toxic gain-of-function or (ii) a gene for which suppression alleviates a pathology. In one embodiment, the target mRNA is an mRNA selected from the group consisting of: progranulin (PGRN), Huntingtin (HTT), Ataxin 2 (ATXN2), Superoxidase dismutase 1 (SOD1), Chromosome 9 open reading frame 72 (C9orf72), Fused in Sarcoma (FUS), microtubule-associated protein tau (MAPT). Leucine-rich repeat kinase 2 (LRKK2), or alpha-synuclein (SCNA), preferably wherein the mRNA is from PGRN.

In one embodiment, the hybrid microRNA targets the 3′ untranslated region (UTR), 5′UTR or coding sequence (CDS) of the target mRNA, preferably wherein the hybrid microRNA targets the CDS of the target mRNA.

In one embodiment, the stem and/or loop sequences are derived from a microRNA that is expressed in the brain, preferably, wherein the microRNA is expressed in neurons.

In one embodiment, the stem sequence is derived from an intronic microRNA.

In a further aspect, the present invention provides a polynucleotide encoding one or more hybrid microRNAs or precursors thereof, preferably wherein the polynucleotide is a DNA.

In one embodiment, the polynucleotide encodes multiple hybrid microRNAs or precursors thereof, preferably wherein the hybrid microRNAs or precursors thereof comprise different stem and/or loop sequences.

In one embodiment, the polynucleotide encodes multiple microRNAs or precursors thereof, preferably wherein the hybrid microRNAs or precursors thereof comprise different guide sequences.

In one embodiment, the polynucleotide encoding a hybrid microRNA or precursor thereof has at least 85%, 90%, 95 or 99% sequence identity to any SEQ ID NO: 115.

In a further aspect, the present invention provides an expression cassette comprising a polynucleotide comprising a promoter sequence and/or a pre-mRNA 3′end cleavage site and/or a polyadenylation Poly (A) signal, preferably wherein the Poly (A) signal comprises, consists or consists essentially of SEQ ID NO: 55-57, or 76-83.

In one embodiment, the polynucleotide sequence encoding the hybrid microRNA or precursor thereof is located 3′ to the pre-mRNA 3′ end cleavage site and/or Poly (A) signal.

In one embodiment the polynucleotide sequence encoding the hybrid hairpin (preferably the 5′ residue of the stem sequence) is positioned between 40 to 1500 residues from the pre-mRNA 3′ end cleavage site and/or Poly (A) signal (preferably from the 3′ residue of the Poly (A) signal), preferably between 50 to 1000, 100 to 500 or 200 to 300 residues from the pre-mRNA 3′ end cleavage site and/or Poly (A) signal.

In a further aspect, the present invention provides a vector comprising a polynucleotide encoding one or more hybrid microRNAs or precursors thereof, or an expression cassette comprising said polynucleotide and further comprising a promoter sequence and/or a pre-mRNA 3′end cleavage site and/or a polyadenylation Poly (A) signal.

In one embodiment, the vector is a lentivirus or adeno-associated virus (AAV), preferably wherein the vector is a self-complementary (sc) and/or single-stranded (ss) AAV.

In a further aspect, the present invention provides a pharmaceutical composition comprising a hybrid microRNA or precursor thereof, polynucleotide, expression cassette, or vector encoding the same, wherein said pharmaceutical composition further comprises one or more pharmaceutically acceptable excipients, diluents, or carriers.

In one embodiment, the pharmaceutical composition further comprises a liposome.

In a further aspect, the present invention provides a method of degrading and/or inhibiting translation of mRNA in a cell, said method comprising contacting the cell with a hybrid microRNA or precursor thereof, a polynucleotide, expression cassette, or vector or pharmaceutical composition as described herein.

In a further aspect, the present invention provides a hybrid microRNA or precursor thereof, polynucleotide, expression cassette, vector or pharmaceutical composition as described herein, for use in reducing or inhibiting the expression of a target polynucleotide in a subject in need thereof.

In a further aspect, the present invention provides a hybrid microRNA or precursor thereof, polynucleotide, expression cassette, vector or pharmaceutical composition as described herein, for use in treating or preventing a neurodegenerative disease in a subject in need thereof.

In one embodiment, the neurodegenerative disease is selected from the group consisting of: Huntington's disease (HD), Parkinson's disease (PD), Fronto-temporal dementia (FTD), Alzheimer's disease (AD), Amyotrophic Lateral Sclerosis (ALS), Creutzfeldt-Jakob disease (CJD), Spinocerebellar Ataxia 2 (SCA2), and progressive supranuclear palsy (PSP).

In a further aspect, the present invention provides a method of treating or preventing a disease, disorder or condition in a subject in need thereof, wherein the method comprises administering to the subject a therapeutically effective amount of the hybrid microRNA or precursor thereof, polynucleotide, expression cassette, vector or pharmaceutical composition as described herein, preferably wherein the disease, disorder or condition is a neurodegenerative disease.

In a further aspect, the present invention provides a method of producing a hybrid microRNA or precursor thereof that suppresses expression of a target mRNA, the method comprising:
(i) selecting, preparing or obtaining a hybrid microRNA backbone sequence, or a polynucleotide, expression cassette or vector that encodes said hybrid microRNA backbone sequence;
wherein the hybrid microRNA backbone sequence comprises a stem sequence from a first intronic microRNA, and a loop sequence from a second microRNA; and
(ii) cloning a guide sequence that binds specifically to a target mRNA into said hybrid microRNA backbone sequence, or into said polynucleotide, expression cassette or vector;
wherein the stem, loop and guide sequences together form a hybrid hairpin that is processed to form a mature microRNA comprising the guide sequence, and the mature microRNA directs degradation of and/or inhibits translation of said target mRNA.

In one embodiment the hybrid microRNA backbone further comprises a pre-mRNA 3′ end cleavage site and/or Poly (A) signal and a promoter.

In one embodiment, the guide sequence comprises 12-30 nucleotide residues.

In one embodiment the stem and/or loop sequences are derived from a microRNA that is expressed at least in the brain, preferably, wherein the microRNA is expressed in neurons.

In one embodiment the stem sequence is derived from an intronic microRNA.

In a further aspect, the present invention provides a kit for cloning a guide sequence into a hybrid microRNA backbone, said kit comprising:
(i) a polynucleotide encoding said hybrid microRNA backbone, comprising:
a stem sequence derived from a first intronic microRNA, and a loop sequence derived from a second microRNA; and
(ii) one or more reagents suitable for cloning a guide sequence that binds specifically to a target mRNA into said polynucleotide;
wherein the stem, loop and guide sequences together form a hybrid hairpin that is processed to form a mature microRNA comprising the guide sequence, and the mature microRNA directs degradation of and/or inhibits translation of said target mRNA.

In one embodiment the hybrid microRNA backbone further comprises a pre-mRNA 3′ end cleavage site and/or Poly (A) signal and a promoter.

In one embodiment, the guide sequence comprises 12-30 nucleotide residues.

In one embodiment the stem and/or loop sequences are derived from a microRNA that is expressed at least in the brain, preferably, wherein the microRNA is expressed in neurons.

In one embodiment the stem sequence is derived from an intronic microRNA.

### BRIEF DESCRIPTION OF THE DRAWINGS

The accompanying drawings are not intended to be drawn to scale. The Figures are illustrative only and are not required for enablement of the disclosure. For purposes of clarity, not every component may be labelled in every drawing.

FIG. 1. A) An illustration of the physiological processing of endogenous miRNA transcribed from the genome. B) Engineered miRNA hybrid designed to be processed in an identical manner to endogenous miRNA.

FIG. 2. Stem-loop hybrid miRNA design for efficient knockdown in neuron. A) Stem Component: miR-423 is located within intron 1 of the NSRP1 gene. The 438 bp intronic sequence, including the miR-423 stem sequence, was used for the vector design. B) Cloning: Type IIS restriction enzymes recognize asymmetric DNA sequences and cleave outside of the recognition site. The recognition sequence (GGTCTC) is 6 nucleotides long and has an overhang length of 4 nucleotides. C) Novel miRNA platform: the miR-423 stem (in grey) and miR-128-2 features (loop and mismatches, in green) are fused to create a completely novel hybrid miRNA platform. The mature miRNA sequence is at the 3′ side of the hairpin (in red), and the passenger sequence at the 5′ side (in blue). Some G-U bonds (light green) are located between the Drosha site and the central bulge.

FIG. 3. Cloning strategy for pMix and guide sequence using “amiR-Plug in” construct. A) BsaI digestion opens compatible DNA sequences on 5′ (AATC) and on 3′ (CTTG) strands. B) Two oligos are synthesised to generate both strands of the insert. The top strand oligo includes the mature miRNA sequence (guide, red bold) and the passenger (blue bold). Each oligos have a 5′ extension (TTAG (SEQ ID NO: 5) and CAAG (SEQ ID NO: 6)) to generate compatible cohesive ends after annealing. This amiR-plug can be directly ligated into to the BsaI digested pMiX stem sequence.

FIG. 4. miRNA expression vector design. A) pMiX1: The miR-423-128 intron and flanking exons are located 5′UTR of EGFP. Note: A stop codon (TAA) is incorporated in the intron to prevent intron retention. B) pMiX2: The miR-243-128 intron splits EGFP with splicing donor and acceptor sites. C) pMiX3: The miR-243-128 intron and flanking exons are located within the 3′ UTR of EGFP. D) pcDNA6.2: The miR-155 flanking sequence from Invitrogen is used as a positive control.

FIG. 5. GFP fluorescence and splicing of pMiXs vector. A) Fluorescence levels: plasmids were transfected into HEK-293 cells and 24 hours later cells were harvested and the GFP fluorescence was measured (data from 4 independent repeats). B) Splicing rate of pMiXs: RT-PCR using flanking primers. pMiXO expresses a GFP without intron and additional exons.

FIG. 6. miRNA activity test using Dual luciferase reporter. A) miRNA vector plasmids were co-transfected with pmiR-Glo DLR plasmid (Promega), which has a miRNA target sequence in the 3′UTR of firefly luciferase. The Firefly luciferase is normalized for transfection efficiency by the Renilla luciferase signal. B) Plasmids were transfected into HEK-293 cells, 24 hours later cells were harvested and the DLR assay is performed by using DLR assay kit (Promega), (data from 4 independent repeats).

FIG. 7. miRNA processing is most efficient when placed after the polyA sequence. A) Schematic diagram of the pA with miRNA. B) DLR assay results decrease in the Firefly luciferase normalised to the Renilla signal. (N=4 experimental replicates).

FIG. 8. Poly A enhance miRNA processing for intronic miRNA hybrid. A) The hairpin of miR-423-128 were cloned before and after the polyA and tested on 3′UTR and 5′UTR target sequence. B) miR-155 based constructs were repositioned before (pMiX6) and after (pMiX6.4) the polyA sequence. These systems were tested on 3′UTR or 5′UTR target sequence of luciferase due to adding nonspecific sequence to CDS will cause mutated luciferase.

FIG. 9. Increasing distance between poly-A and hairpin reduces miRNA activity. A) Schematic diagram of various distances of the miRNA hairpin from polyA. Scrambled sequences were added to stretch the sequence between PAS and hairpin. B) DLR assay for the constructs. (N=3 experimental replicates).

FIG. 10. pMiX miRNAs are more efficiently processed at earlier time points. Plasmids were transfected into HEK-293 and cells were harvested 8, 16 and 24 hours later. The DLR assay shows that pMiX2 and pMiX4 are processed rapidly and effect a significantly greater knock down than pcDNA6.2 at 8 and 16 hours (N=3 experimental replicates). Adjusted P values obtained from Tukey's multiple comparisons test are listed on the right for the different time points, 8, 16 and 24 hours.

FIG. 11. Both pMiX2 and pMiX4 efficiently silence the 5′UTR and CDS mRNA. A) Schematic diagram of the Firefly reporter gene for 5′UTR, CDS and 3′UTR with a miR128 target sequence were cloned into the 5′ or 3′UTR of Firefly in the DLR vector. B) Luciferase activity recorded 24 hours following transfection of HEK-293 cells with pMiX2, pMiX4 and pcDNA6.2 three constructs. pMiX 4 achieved the greatest reduction in luciferase activity when targeting the 5′UTR, CDS and 3′UTR. C) Firefly luciferase mRNA was quantified by qPCR to determine whether the decrease in Luciferase activity observed was due to suppression of translation or mRNA degradation. pMiX 4 achieved the greatest reduction in luciferase signal and mRNA levels when targeting the 5′/UTR, CDS or 3′UTR. (N=3 experimental replicates).

FIG. 12. Quantification of Guide and Passenger miRNA for different constructs. A) miR-Fluc1 and miR-128 Guide strands were quantified by RTqPCR from pcDNA6.2, pMiX2 and pMix4 24 hours after HEK-293 cell transfection. B) Guide and passenger miRNAs expression: the unwanted expression of the Passenger strand was quantified compared the total expression of both strands, from pcDNA6.2, pMiX2 and pMiX4.

FIG. 13. Effect of Drosha knockdown on miRNA efficiency. A) Drosha or control (CONT) siRNAs are transfected the first day in HEK 293 cells. DLR reporter and miRNA vectors are transfected after 24 hours. DLR assays are carried out after 24 and 48 hours of additional incubation. B) Drosha knockdown is confirmed by RT-qPCR at each time point. C) DLR assays show that pMiX4 silencing is highly efficient and unaffected by Drosha knock-down. (N=2 experimental replicates).

FIG. 14. pMix4 knocks down PGRN expression in a sequence-specific manner. PGRN wildtype (PGRN-WT) and codon optimised (PGRN-CO) plasmids were co transfected with pMiX2-GRN and pMiX4-GRN selectively targeting the PGRN-WT sequence which differed with PGRN-CO by only 2 base pairs. PGRN protein levels were quantified by Western blot (WB) from (A) Cell lysate (normalised to GAPDH) and (B) Supernatant (normalized to Transferrin) demonstrated a 75% reduction in protein while PGRN expression driven by the PGRN-CO construct was unaffected.

FIG. 15. Contribution of intronic elements to miR-423 knockdown efficacy. A) Schematic diagram of miR-423-128 RNA secondary structure. Black line indicates deletion mutants for pMiX (4.1, 4.2, 4.3 and 4.4). B) DLR assay. C) Intron and stem sequence of miR-423.

FIG. 16. Schematic diagram of SR protein binding site on pMiX. The intronic sequence of the pMiX variants were screened for SR binding sites by “ESE finder” software (http://krainer01.cshl.edu/cgi-bin/tools/ESE3/esefinder.cgi?process=home).

FIG. 17. ESE Finder identified several SR binding sites in pMIX intronic sequences. The sequences indicating for the SR protein binding site. A) SR binding site for pMiX4.1. B) SR binding site for pMiX4.2.

FIG. 18. Other intronic hybrid miRNAs showing equivalent efficacy to pMIX4. We added intronic miRNAs (miR-26, miR-126, and miRNA cluster-106b, 93 and miR-25) to test the knockdown efficiency. The pMIX-423 is used for the comparison of DLR activity.

FIG. 19. Self-complementary (scAAV) and single stranded (ssAAV) pMix4 plasmids. A) Plasmid map of scAAV-iMix4. GFP expressed under CMV promoter, and the miRNA is located after the HSV TK polyA signal. ITR-ITR size is 2935 bp. B) Plasmid map of ssAAV-iMIX4. GFP expressed under CB7 promoter, and the miRNA is located after the HSVTK polyA signal. ITR-ITR size is 3335 bp.

FIG. 20. ATXN2 knockdown with sc-iMiX and ss-iMiX system. ITR plasmids that has self-complementary (sc) or single stranded (ss) miRNA against ATXN2 transfected to HEK-293 (A), HeLa (B), C2C12 (C) and rat primary cortical neurons (D). DLR assay performed for sc-iMiX group (Control, miR-ATXN2) and ss-iMiX group (Control, miR-ATXN2). N=3.

FIG. 21. ATXN2 AAV vMiX knockdown test by in vivo and in vitro. AAV serotype 1 is used for packaging the scMIX and ssMIX to transduce broad type of cell lines to test the efficacy of the virus. A) Alkaline gel for the self-complementary and single stranded harbouring the miRNA hairpin. B) Capsid packaging. C) sc-vMiX and ss-vMIX AAVs were transduced to HEK-293 cells for DLR assay. D) RT-qPCR assay for endogenous ATXN2 mRNA expression level, which normalized by GAPDH.

### DETAILED DESCRIPTION OF THE INVENTION

Unless otherwise defined below, all technical terms used herein have the same meaning as commonly understood by one of the ordinary skill in the art in the field to which this disclosure belongs.

Disclosed herein is a novel miRNA cloning system comprising a hybrid microRNA comprising stem and loop sequences from different microRNAs. For instance, the hybrid microRNA may comprise a stem sequence from a first microRNA, a loop sequence from a second microRNA, and a guide sequence that binds to a target mRNA. Preferably the stem sequences are derived from intronic microRNAs. The present disclosure also relates to precursors of said hybrid, polynucleotides encoding said hybrid microRNA or precursors thereof, expression cassettes, vectors, complexes or compositions comprising the polynucleotide, hybrid miRNA or precursors thereof.

The hybrid microRNA of the present invention surprisingly overcomes one or more of the problems encountered when using existing cloning systems (e.g. the commercial miRNA cloning system pcDNA6.2). For instance, the present hybrid microRNA may provide a high rate of knockdown of a target gene. In one embodiment, it was found that the presently disclosed hybrid microRNA allows at least an increased rate of knockdown compared to pcDNA6.2 at 8 and 16 hours.

In addition, the present hybrid microRNA may increase knockdown efficiency compared to existing cloning systems, particularly when targeting the 5′ UTR or CDS. For instance, the presently disclosed hybrid microRNA of one embodiment resulted in a knockdown efficiency of up to 80% when targeting a sequence in the 5′ UTR of an mRNA compared to 38% when targeting a sequence in the 5′ UTR of an mRNA using pcDNA6.2. In addition, the presently disclosed hybrid microRNA of one embodiment resulted in highly efficient knockdown of mRNA when targeting the CDS of an mRNA compared to the pcDNA6.2 construct. For instance, the presently disclosed hybrid microRNA of one embodiment resulted in a knockdown efficiency of up to 80% when targeting a sequence in the CDS of an mRNA compared to 30% when targeting a sequence in the CDS of an mRNA when using pcDNA6.2.

The presently disclosed hybrid microRNA may also provide an increased knockdown efficiency (e.g. compared to existing cloning systems) when targeting the 3′ UTR of a gene. For instance, in one embodiment the present hybrid microRNA resulted in a knockdown of up to 80% when targeting a sequence in the 3′ UTR of an mRNA, compared to around 65% knockdown when targeting a sequence in the 3′ UTR of an mRNA when using pcDNA6.2.

In addition, the presently disclosed hybrid microRNA may also result in an improved ratio of guide miRNA strands to passenger miRNA strands compared to known microRNA cloning systems. For instance, the present hybrid microRNA may lead to a reduced proportion of passenger strands (and thus an increased proportion of guide microRNA strands) being loaded into the RISC, thereby reducing off-target effects. For instance, one embodiment of the present hybrid microRNA results in around 0-7% of strands being loaded into the RISC being the passenger strand, compared to 20-25% for pcDNA6.2. Therefore, the present hybrid microRNA also results in reduced off-target effects compared to the commercial pcDNA6.2 construct.

It was surprising that using the stem sequence from one miRNA (e.g., miR-423) and the loop from another (e.g., miR-128-2) could result in one or more of the aforementioned improvements over commercial miRNA cloning systems, e.g. pcDNA6.2. Furthermore, it was unexpected that positioning a miRNA sequence downstream of (3′ to) a 3′ end pre-mRNA cleavage site and/or poly (A) site could result in more efficient knockdown of the target mRNA.

### General Definitions

Any reference to ‘or’ herein is intended to encompass “and/or” unless otherwise stated.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

The terms “reduce” “suppress”, “inhibit”, “decrease” or “knockdown” are all used interchangeably herein to mean that something is lessened, preferably by a statistically significant degree.

### DNA/RNA

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as rare nucleosides).

The term “nucleotide” or “nucleotide residues” refers to any nucleoside having one or more phosphate groups joined in ester linkages to the sugar moiety. Exemplary nucleotides include nucleoside monophosphates, diphosphates and triphosphates. The term nucleotide is used generally to refer to nucleotides found in DNA and RNA; adenine (A), guanine (G), cytosine (C), thymine (T) and uracil (U). The terms “polynucleotide” or “nucleic acid molecule” are used interchangeably herein and refer to a polymer of nucleotides joined together by a phosphodiester or phosphorothioate linkage between 5′ and 3′ carbon atoms, including DNA and RNA. As used herein, the term “DNA” or “deoxyribonucleic acid” is used to specifically refer to a polymer of A. T. C and G nucleotides. As used herein, the terms “ribonucleic acid molecule”, or “RNA” are used to refer to polymers of A, U, C or G nucleotides. A “DNA” or “RNA”, as referred to herein, may be any length, including at least 2, 3, 4, 5, 10, 15, 20, 25, 30, 50, 100, 1000, 10,000 or more nucleotides. The DNA or RNA may be endogenous, exogenous or artificial. The DNA or RNA may be double or single stranded.

The term “gene” is used in line with the art. Briefly, “gene” refers to a basic unit of DNA sequence which may instructions for a specific molecule such as RNA or a protein. RNA or DNA sequences may also comprise “introns” or “exons”. “Exons” or “exonic sequences” are sequences that code the final polypeptide sequence, whereas “introns” or “intronic sequences” are sequences within an RNA or DNA sequence which may be considered not to code or a protein. Introns generally are removed from precursor messenger mRNA (pre-mRNA) by RNA splicing.

The term “sequence” refers to any contiguous string of nucleotides, such as a DNA or RNA sequence. A “sequence” may be at least 2, 5, 10, 15, 20, 30, 40, 50, 100, 200, 500, 1000 nucleotides in length.

In embodiments, the present nucleotide sequences may be modified to replace the intended RNA or DNA nucleotide with “nucleotide analogues”, “modified nucleotides” or “altered nucleotides” which are non-standard, non-naturally occurring ribonucleotides or deoxyribonucleotides. Exemplary nucleotide analogues are modified at any position so as to alter certain chemical properties of the nucleotide yet retain the ability of the nucleotide analogue to perform its intended function. In addition, the phosphate group of the nucleotide may be modified by making substitutions which still allow the nucleotide to perform its intended function. These have been described extensively in the art and are very well known to a skilled person.

In embodiments, the RNA may be a single-stranded messenger RNA (“mRNA”). mRNA is produced by transcribing DNA to form pre-mRNA, and then further processing the pre-mRNA to produce mature mRNA. The mature mRNA may then be translated into amino acid sequences (“polypeptides” or “proteins”), as each three nucleotides (also known as a “codon”) encodes a specific amino acid. mRNA can be post transcriptionally modified, including constitutive and alternative splicing, performed by the spliceosome. “Constitutive splicing” is the process of removing introns from the pre-mRNA, and joining the exons together to form a mature mRNA. Alternative splicing, on the other hand, is the process where exons can be included or excluded in different combinations to create a diverse array of mRNA transcripts from a single pre-mRNA and therefore serves as a process to increase the diversity of the transcriptome. There are four modes of alternative splicing: (1) exon skipping, (2) mutually exclusive exon usage, (3) alternative splice site selection, and (4) intron retention. Exon skipping (1) denotes the excision of ≥1 exons and its surrounding introns from a pre-mRNA. Mutually exclusive exon usage (2) represents a form of exon skipping where either one or another exon, but never both, is included in the mature mRNA. Alternative splice site selection (3) relies on the possibility of using different splice sites at the 5′ and 3′ end of an exon, resulting in longer or shorter exons from the same transcript. Intron retention (4) is a process in which (part of) an intron is retained in the mature mRNA transcript. Intronic or exonic splicing enhancers are short (between 3-10 nucleotides) polynucleotide sequences which assist in the recruitment of splicing factors to facilitate the splicing of pre-mRNA to form mRNA.

As used herein, the term “base pair” refers to the interaction between pairs of nucleotides (or nucleotide analogs) on opposing strands of nucleotide sequences (e.g., a duplex formed by a strand of a guide RNA and a target RNA sequence), due primarily to Hydrogen bonding, van der Waals interactions, and the like between said nucleotides (or nucleotide analogs). “Base pair” or “base pairs” may also refer to the length of a polynucleotide by indicating the number of nucleotides, “base pairs” or “bp”.

The term “expression” has its meaning as understood in the art and refers to the process of converting genetic information encoded in a DNA sequence (“coding sequence”) into RNA (e.g., mRNA, rRNA, tRNA, or snRNA) through “transcription” of a polynucleotide (e.g., via the enzymatic action of an RNA polymerase), and for polypeptide-encoding polynucleotides, into a polypeptide through “translation” of mRNA.

Variants of the nucleotide sequences described herein may also be used in the present invention. For instance, in specific embodiments, the present invention may involve variants of the disclosed stem, loop, guide or hybrid microRNA sequences, precursors thereof or polynucleotides encoding the same. The similarity between nucleotide sequences is expressed in terms of the similarity between the sequences, otherwise referred to as “sequence identity”. Sequence identity is frequently measured in terms of percentage identity (or similarity or homology); the higher the percentage, the more similar the two sequences are. Homologs or variants of the nucleotide sequence will possess a relatively high degree of sequence identity when aligned using standard methods. Methods of alignment of sequences for comparison are well known in the art. As such, variants of the presently disclosed stem, loop, guide or hybrid miRNA sequences, precursors thereof or polynucleotides encoding the same are envisaged, including variants that have at least 70%, 75%, 80%, 85%, 90%, 91%, 92%, 93%, 94%, 95%, 96%, 97%, 98%, or 99% sequence identity with the original sequence (e.g., a sequence defined herein), for example counted over at least 20, 50, 100, 200 or 500 nucleotide residues or over the full length alignment using the NCBI Blast 2.0. gapped blastp set to default parameters. For comparisons of nucleotide sequences of greater than about 30 nucleotides or amino acids, the Blast 2 sequences function is employed using the default BLOSUM62 matrix set to default parameters, (gap existence cost of 11, and a per residue gap cost of 1). When aligning short nucleotides (fewer than around 30 residues), the alignment should be performed using the Blast 2 sequences function, employing the PAM30 matrix set to default parameters (open gap 9, extension gap 1 penalties). Polynucleotides with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 residues, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only: it is entirely possible that strongly significant homologs could be obtained that fall outside of the ranges provided.

“Bind” as used herein can refer to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions are generally characterized by a dissociation constant (Kd) of less than 10⁻³ M, less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M or less than 10⁻¹⁵ M. Kd is dependent on environmental conditions, e.g., pH and temperature, as is known by those in the art. “Affinity” can refer to the strength of binding, and increased binding affinity is correlated with a lower Kd. Thus, the terms “binds to”, “associates with” and “forms a complex with” may be used interchangeably herein. For instance, the mature microRNA in the RISC complex.

The terms “hybridizing” or “hybridize” can refer to the pairing of substantially complementary or complementary nucleic acid sequences within two different molecules. Pairing can be achieved by any process in which a nucleic acid sequence joins with a partially, substantially or fully complementary sequence through base pairing to form a hybridization complex. For purposes of hybridization, two nucleic acid sequences or segments of sequences are “substantially complementary” if at least 80% of their individual bases are complementary to one another. Two nucleic acid sequences or segments of sequences are “partially complementary” if at least 50% of their individual bases are complementary to one another.

Low stringency hybridization refers to conditions equivalent to hybridization in 10% formamide, 5x Denhardt's solution, 6x SSPE, 0.2% SDS at 22° C., followed by washing in 1x SSPE, 0.2% SDS, at 37° C. Denhardt's solution contains 1% Ficoll, 1% polyvinylpyrolidone, and 1% bovine serum albumin (BSA). 20x SSPE (sodium chloride, sodium phosphate, ethylene diamide tetraacetic acid (EDTA)) contains 3 M sodium chloride, 0.2 M sodium phosphate, and 0.025 M (EDTA). Other suitable moderate stringency and high stringency hybridization buffers and conditions are well known to those of skill in the art.

As used herein, “complementary” is used herein in accordance with its art-accepted meaning to refer to the capacity for pairing via hydrogen bonds (e.g., Watson-Crick base pairing or Hoogsteen base pairing) between two nucleosides, nucleotides or nucleic acids. For example, if a nucleotide at a certain position of a first nucleic acid is capable of stably hydrogen bonding with a nucleotide located opposite to that nucleotide in a second nucleic acid, when the nucleic acids are aligned in opposite 5′ to 3′ orientation (i.e., in anti-parallel orientation), then the nucleic acids are considered to be complementary at that position. Complementary nucleotides in DNA are A and T, and C and G, and in RNA it is A and U, and C and G. “Non-complementary” or “mismatch” are used to refer to base pairs, such as T and C, or C and C. “Complementary” may also refer to the complementarity of the bases across the sequence. As such, “complementary” as used herein may refer to nucleic acid sequences that have at least 50% sequence identity. Preferably, the two nucleic acid sequences have at least 60%, 70%, 80%, 90%, 95%, 96%, 97%, 98%, 99%, or 100% of sequence identity. In embodiments, the hybrid microRNA or precursor thereof or guide sequence may be complementary to a nucleotide sequence of between 2 to 30 nucleotides in the target sequence. In some instances, the region of precise sequence complementarity is interrupted by a bulge. See, Ruvkun (2001) Science 294: 797-799. “Complementary” also means that two nucleic acid sequences can hybridize under low, middle, and/or high stringency condition(s).

As used herein, “reverse complement” or “reverse complementary” refers to polynucleotide sequences formed by first reversing a sequence, and then inverting the bases to the corresponding base pair (A to T or U, and C to G, and vice versa). For example, ACCTGAG becomes CTCAGGT,

“CDS” or “coding sequence”, as used herein, refers to the sequence of mRNA or DNA that encodes the final polypeptide (or “protein”).

“UTR” or “untranslated regions”. as used herein, refers to an untranslated region on a strand of mRNA. There are two UTRs on an mRNA on each side of the coding sequence. If the UTR is found on the 5′ side, it is called the “5′ UTR” (or “leader sequence”), or if the UTR is found on the 3′ side, it is called the “3′ UTR” (or “trailer sequence”). Within the 5′ UTR is a sequence that is recognized by the ribosome which allows the ribosome to bind and initiate translation. In contrast, the 3′ UTR plays a role in translation termination and post-transcriptional modifications of the mRNA. The UTRs are different to introns in that they are found in the final mRNA, whereas introns are spliced out of the mRNA and are therefore considered “non-coding”.

### MicroRNA

microRNAs are non-coding RNAs that regulate gene expression by directing target messenger RNA for degradation or translational inhibition. microRNA genes are mostly located in non-coding gene regions, although others may be located in an intron or untranslated region (UTR) of a protein coding gene. As used herein, the terms “microRNA” or “miRNA” refers generally to both the mature microRNA or precursors thereof, including endogenous, exogenous and artificial miRNAs. In embodiments, the term microRNA or miRNA may include the presently disclosed hybrid microRNA or precursors thereof.

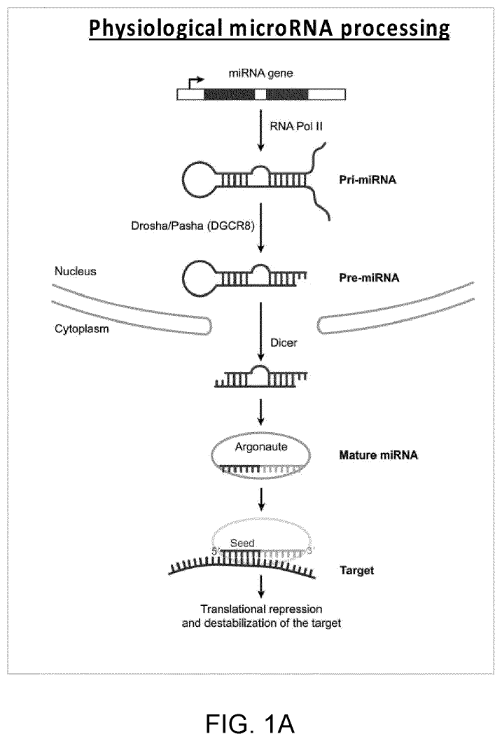

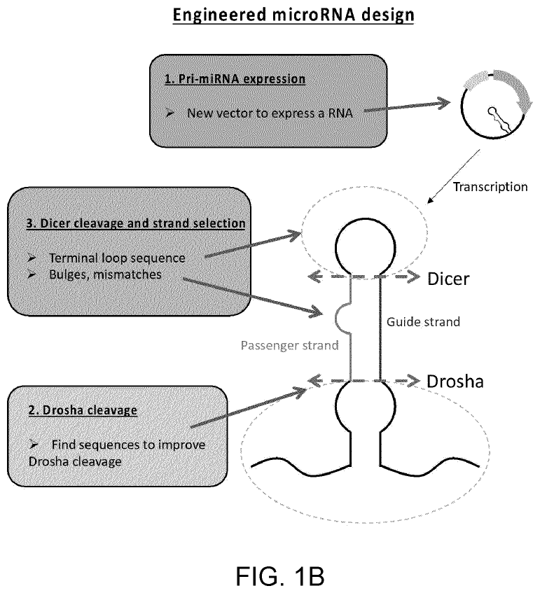

microRNAs are generated through a series of steps (see FIG. 1A) beginning with the transcription of a primary miRNA transcript (“pri-miRNA”) in the nucleus from DNA. As such “pri-mRNA” refers to the with a stem-loop structure and may be up to thousands of nucleotides in length. The pri-mRNA is then processed in the nucleus by cleavage by RNase III enzyme Drosha (FIG. 1A). Drosha is a protein of approximately 160 kDa in size with two tandem RNase III domains (RIIIDs) and a double-stranded RNA-binding domain (dsRBD) that are all necessary for catalysis. The region of the protein adajacent to the RIIIDs is also essential for pri-miRNA processing. Drosha is found in a large complex (approximately 500-650 kDa) called the Microprocessor complex. Drosha cleaves the pri-mRNA at sites near the base of the primary stem loop to release the double stranded stem loop structure, referred to as “precursor microRNA” (see FIG. 1A). As such, “precursor microRNA”, “precursor miRNA” or “pre-miRNA” refers to a polynucleotide sequence with a stem-loop structure which is between 50-100 nucleotides, typically 70 nucleotides in length. However, as used herein, the term “microRNA precursors” or “miRNA precursors” are used to refer generally to any precursor sequence of the mature microRNA, including, but not limited to, primary microRNA (pri-miRNA), a pre-microRNA (pre-miRNA), or a transcript comprising a microRNA that has been encoded by a polynucleotide, expression cassette or vector.

Pre-miRNAs are then exported from the nucleus to the cytoplasm by exportin-5, where the pre-miRNAs are further cleaved into double stranded miRNA sequences, referred to as a “microRNA duplex” (see FIG. 1A) by the cytoplasmic RNase III enzyme “Dicer”. Therefore, the term “microRNA duplex” as used herein refers to the double stranded, Drosha/Dicer processed microRNA which comprises both the mature microRNA strand and the passenger strand. Dicer is a protein of about 200 kDa, comprising two RIIIDs, a dsRBD, and an N-terminal segment comprising a DEAD-box RNA helicase domain, a DUF283 domain, and a PAZ domain. PAZ domains bind to the 3′ overhanging end of small RNAs, such as the short (about 2 nucleotide) 3′ overhang on pre-miRNAs created by Drosha cleavage of the pri-miRNA transcript. The microRNA duplex comprises two strands; the “mature microRNA” strand which is the strand that will bind to the complementary (fully or partially) target sequence, and the microRNA*strand” or “passenger strand” which is complementary to the microRNA itself (fully or partially). Therefore, as used herein, “mature microRNA”, “mature miRNA” or “guide” are used to refer to the non-coding, single-stranded RNA molecule that is e.g. between 17 to 27 nucleotides in length (including 17, 18, 20, 21, 22, 23, 24, 25, 26, or 27 nucleotides in length). As used herein, the terms “* strand”, “star strand” or “passenger strand” are used interchangeably to refer to the non-coding, single stranded RNA molecule which is partially or fully complementary to the mature microRNA strand.

One of the strands from the microRNA duplex is then incorporated into RNA-induced silencing complexes (RISC) with the Argonaute (AGO) protein. The strand that is chosen by RISC to direct the degradation or translational inhibition of a complementary target mRNA is dependent upon the degree of stability of the termini of the miRNA duplex. The strand with lower stability base pairing of the 2-4 nucleotides at the 5′ end of the duplex preferentially associates with RISC and thus, becomes the mature miRNA (Schwarz et al. (2003) Cell 115(2): 199-208). Therefore, the mature microRNA may be incorporated into the RISC and guide the RISC to complementary (partially or fully) RNA molecules, wherein the RISC either degrades the target mRNA or blocks the translation of the target mRNA to a polypeptide.

Therefore, as used herein, the term “processed” or “processing” of microRNA may refer to cleavage of precursor microRNAs by Drosha and/or Dicer, or the incorporation of the mature microRNA or passenger microRNA strands into the RISC.

As used herein, microRNA names (denoted by mir-# or miR-#) are used interchangeably to refer to the microRNA sequence of interest. If specifically noted, the uncapitalized “mir” prefix generally refers to the pre-miRNA, while a capitalized “miR” prefix usually refers to the mature form.

As used herein, the terms “stem-loop structure” or “hairpin” are used interchangeably to refer to a polynucleotide with a predicted or known secondary structure where, two sequences that are partially or fully complementary when read in opposite directions, base pair (referred to as a “stem”), leaving an unpaired sequence at the end which forms a “loop” between the base-paired region. Therefore, as referred to herein, “stem portion”, “stem region” or “stem sequence” refers to a polynucleotide sequence that may contribute to the stem region of a hairpin. As referred to herein, the terms “loop portion”, “loop region” or “loop sequence” refer interchangeably to the polynucleotide sequence which is known, or is predicted to form a loop sequence in a hairpin. In embodiments, the terms “stem-loop structure” or “hairpin” refer to a pre-microRNA or pri-microRNA comprising the stem and loop. In preferred embodiments, the terms “stem-loop structure” or “hairpin” refer to a hybrid pre-microRNA or pri-microRNA sequence, or a polynucleotide encoding the same.

### Hybrid MicroRNA

As used herein, the terms “commercial vector”, “commercial platform” or “pCDNA 6.2” are used interchangeably to refer to, for example, the pcDNA6.2-GW/EMGFP-miR (BLOCK-ItTM, Thermofisher) microRNA cloning system.

As used herein, the term “hybrid microRNA”, “hybrid miRNA”, “hybrid hairpin”, or “miRNA platform” refers to a mature microRNA, stem-loop structure, hairpin, microRNA duplex, pre-miRNA, pre-miRNA, precursor, or a polynucleotide encoding the same, which comprises the stem sequence of a first microRNA, the loop sequence of a second microRNA and a guide sequence. In preferred embodiments, the second microRNA is a different microRNA to the first. In embodiments, the stem sequence and/or loop sequence is derived from a miRNA that is known, or predicted, to be expressed in the central nervous system, preferably the brain. In specific embodiments, the stem and/or loop sequence is known, or predicted, to be expressed in neurons. In embodiments, the stem sequence is derived from an intronic microRNA. In embodiments, the loop sequence is derived from an intronic microRNA.

In embodiments, the stem sequence of the hybrid microRNA or precursor thereof is derived from one of the following microRNAs: miR-423 (SEQ ID NO: 49), miR-26b (SEQ ID NO: 50), miR-126 (SEQ ID NO: 51), miR-106b (SEQ ID NO: 52), miR-93 (SEQ ID NO: 53), or miR-25 (SEQ ID NO: 54). In embodiments, the 5′ stem sequence of the hybrid microRNA or precursor thereof comprises any one or more of SEQ ID NOs: 19, and 58-62. In preferred embodiments, the 5′ stem sequence of the hybrid microRNA or precursor thereof has at least 60%, 65%, 70%, 75%, 80%, 82%, 85%, 87%, 90%, 91%, 92%, 93%, 94%, 95%, 96%, 97%, 98%, 99% or 100% homology (e.g. sequence identity) to any one or more of SEQ ID NOs: 19, and 58-62. In embodiments, the 3′ stem sequence of the hybrid microRNA or precursor thereof comprises any one or more of SEQ ID NOs: 20 and 63-67. In preferred embodiments, the 3′ stem sequence of the hybrid microRNA or precursor thereof has at least 60%, 65%, 70%, 75%, 80%, 82%, 85%, 87%, 90%, 91%, 92%, 93%, 94%, 95%, 96%, 97%, 98%, 99% or 100% homology (e.g. sequence identity) to any one or more of SEQ ID NOs: 20 and 63-67. As used herein (including with reference to the SEQ ID NOs above and below), sequences may be defined as DNA or the corresponding RNA equivalent. Thus, the microRNAs (including stem, loop and guide sequences thereof) described herein may comprise an RNA sequence corresponding to a DNA sequence as defined in one or more of the relevant SEQ ID NOs, i.e., wherein T residues are replaced with U.

In embodiments, the loop sequence of the hybrid microRNA or precursor thereof is derived from one of the following microRNAs: miR-128-2 (SEQ ID NO: 69), miR-423 (SEQ ID NO: 49), miR-26b (SEQ ID NO: 50), miR-126 (SEQ ID NO: 51), miR-106b (SEQ ID NO: 52), miR-93 (SEQ ID NO: 53), or miR-25 (SEQ ID NO: 54. In embodiments, the loop sequence of the hybrid microRNA or precursor thereof comprises any one or more of SEQ ID NOs: 2 and 70-75. In preferred embodiments, the loop sequence of the hybrid microRNA or precursor thereof has at least 60%, 65%, 70%, 75%, 80%, 82%, 85%, 87%, 90%, 91%, 92%, 93%, 94%, 95%, 96%, 97%, 98%, 99% or 100% homology (e.g. sequence identity) to any one or more of SEQ ID NOs: 2 and 70-75.

In some embodiments, the hybrid microRNA or precursor thereof is a pre-microRNA or pri-microRNA. Such a pri-microRNA may, for example, further comprise an intronic sequence 5′ to the stem and loop sequences. It is particularly preferred that the 5′ intronic sequence is associated with the same intronic miRNA as the stem sequence, e.g. miR-423. Alternatively, the 5′ intronic sequence may be derived from an intron that encodes miR-26b, miR-126, miR-106b, miR-93 or miR-25. For instance, the 5′ intronic sequence may comprise or consist of all or part of SEQ ID NO: 18, or a sequence having at least 85%, 90%, 95% or 99% sequence identity to SEQ ID NO: 18. Alternatively, the 5′ intronic sequence may comprise or consist of all or part of any one of SEQ ID NO: 84-88, or a sequence having at least 85%, 90%, 95% or 99% sequence identity to any one of SEQ ID NO: 84-88.

In a similar way, it is preferred that the hybrid microRNA or precursor comprises an intronic sequence 3′ to the stem and loop sequences, preferably derived from the same intronic miRNA as the stem sequence. Thus the 3′ intronic sequence may be derived e.g. from miR-423, miR-26b, miR-126, miR-106b, miR-93 or miR-25, preferably miR-423. More preferably the 3′ intronic sequence comprises or consists of SEQ ID NO: 21 or any one of SEQ ID Nos 89-93, or a sequence having at least 85%, 90%, 95% or 99% sequence identity to SEQ ID NO: 21 or any one of SEQ ID Nos 89-93.

It is also preferred that the 5′ or 3′ intronic sequence in the hybrid microRNA precursor (e.g. pri-microRNA) comprises a significant part of the endogenous intron, e.g. of the intronic sequence from which the stem sequence is derived. Thus the 3′ or 5′ intronic sequence in the hybrid microRNA precursor may comprise at least 30, 50, 100, 150 or 200 nucleotide residues of intronic sequence (e.g. of any one of SEQ ID NO: 18, 21, 84-88 or 89-93). More preferably the 5′ intronic sequence comprises 50 to 250 or 100 to 250 nucleotide residues of, or at least 50, 100, 150 or 200 nucleotide residues of, or the full length of SEQ ID NO: 18, 21 or any one of SEQ ID Nos 84-93. In some embodiments, the hybrid microRNA precursor comprises a sequence as defined in any one of SEQ ID Nos 7 or 14-17, preferably SEQ ID NO: 7 or 14.

As used herein, the terms “target” or “target sequence” are used interchangeably to refer to any endogenous or exogenous, sense or antisense polynucleotide where cleavage or inhibition of translation is desired or intended. In embodiments, the hybrid microRNA of the present disclosure inhibits the translation of, or results in the degradation of the target sequence. In preferred embodiments, the target polynucleotide is an RNA, preferably an mRNA. In preferred embodiments, the target sequence is within the 3′ UTR, 5′ UTR or CDS of an mRNA. In embodiments, the target polynucleotide is associated with the initiation, progression or maintenance of a pathological state. In embodiments, the target polynucleotide sequence is an mRNA of one or more of the following genes: Progranulin (PGRN), Huntington (HTT), Ataxin 2 (ATXN2), Superoxidase dismutase 1 (SOD1), chromosome 9 open reading frame (C9orf72), Fused in Sarcoma (FUS), microtubule associated protein tau (MAPT), leucine-rich repeat kinase 2 (LRKK2), alpha-synuclein (SCNA), dystrophin (DMD).

As used herein, the term “guide sequence”, or “guide” refers to any polynucleotide sequence with sufficient complementarity to the target polynucleotide sequence. In embodiments, the guide sequence is a mature microRNA sequence. In embodiments, the guide sequence is between 15-40, 20-40, 15-35, 20-35, 15-30, 20-30 or 22-30 nucleotides in length. In preferred embodiments, the guide sequence is equal to or more than 17, 18, 19, 20, 21, 22, 23, 24, 25, 26 or 27 nucleotides in length. In some embodiments, the degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, such as Clustal W or BLAST. In embodiments, the guide sequence may be an existing microRNA sequence, or an artificially selected sequence that has sufficient complementarity to any region of the intended target mRNA. In embodiments, the guide sequence may have sufficient complementarity to a target a sequence in the 3′ UTR of a target mRNA, the 5′ UTR of a target mRNA, the coding sequence of the target mRNA or promoter regions (Xu et al. (2014). Identifying microRNA targets in different gene regions. BMC Bioinformatics 15(Suppl. 7):S4).

As used herein, the term “sufficient complementarity” means any RNA (including microRNA) sequence or precursor which would allow either binding to the target sequence or direct the RISC complex to the target sequence.

As used herein, the terms “seed region” or “seed sequence” are used to refer to the sequence from the second nucleotide to the eighth nucleotide at the 5′ end of the mature microRNA sequence. This “seed sequence” is thought to be crucial for target recognition.

In embodiments, the “hybrid microRNA” also comprises a passenger sequence. In the hybrid microRNA or precursor thereof, the passenger strand is partially or fully complementary to the guide sequence. In preferred embodiments, the passenger strand has some mismatches to the guide sequence in order to generate a central bulge in the hybrid microRNA or precursor thereof. In embodiments, the passenger sequence has at least 60%, 65%, 70%, 75%, 80%, 82%, 85%, 87%, 90%, 91%, 92%, 93%, 94%, 95%, 96%, 97%, 98%, 99% or 100% sequence identity to the reverse complement of the guide sequence.

Exonic splicing enhancers (ESE) play a role in both alternative and constitutive splicing, and many ESEs act as binding sites for members of the Serine/Arginine (Ser/Arg or SR) proteins. Inhibition of mRNA activity by the hybrid microRNA or precursor thereof may be dependent on “SR binding sites”. Therefore. in embodiments, the hybrid microRNA or precursor thereof comprises one or more of the following 5′ SR binding sites: SRSF5-(TTTCCCG (SEQ ID NO: 22), TTTGAGG (SEQ ID NO: 23)) SRSF1-(CGGATGG (SEQ ID NO: 24), AGCCCGA (SEQ ID NO 25), SRSF1-HMG (CGGATGG (SEQ ID NO: 26)), SRSF-(AACTTGTG (SEQ ID NO: 27)), SRSF1-(GTGAGGA (SEQ ID NO: 36), SRSF6-(TGAGGA (SEQ ID NO: 37)), SRSF5-(ATAAAGG (SEQ ID NO: 38)). In embodiments, the hybrid microRNA or precursor thereof comprises one or more of the following 3′ SR binding sites: SRSF6-(TGAGTA (SEQ ID NO: 28)), SRSF5-(TTTCTCC (SEQ ID NO: 29), TCTCAGG (SEQ ID NO: 30)), SRSF1-HMG-(CTCCCCG (SEQ ID NO: 31), CCCCGCT (SEQ ID NO: 32), and CTCAGGG (SEQ ID NO: 33)), SRSF1-(CTCAGGG (SEQ ID NO: 34)), SRSF2-(GGGCAGTG (SEQ ID NO: 35)), SRSF6-(TGCTTC (SEQ ID NO: 39)), or SRSF2-(GCTTCCTA (SEQ ID NO: 40), AACCCGCG (SEQ ID NO: 41)). In embodiments, the hybrid microRNA or precursor thereof comprises at least one or more of the following 5′ SR binding sites: SRSF5-(TTTCCCG (SEQ ID NO: 22), TTTGAGG (SEQ ID NO: 23)) SRSF1-(CGGATGG (SEQ ID NO: 24), AGCCCGA (SEQ ID NO 25)), SRSF1-HMG (CGGATGG (SEQ ID NO: 26)), SRSF-(AACTTGTG (SEQ ID NO: 27)). In embodiments, the hybrid microRNA or precursor thereof comprises at least one or more of the following 3′ SR binding sites: SRSF6-(TGAGTA (SEQ ID NO: 28)), SRSF5-(TTTCTCC (SEQ ID NO: 29), TCTCAGG (SEQ ID NO: 30)), SRSF1-HMG-(CTCCCCG (SEQ ID NO: 31), CCCCGCT (SEQ ID NO: 32), and CTCAGGG (SEQ ID NO: 33)), SRSF1-(CTCAGGG (SEQ ID NO: 34)), SRSF2-(GGGCAGTG (SEQ IDNO: 35)). In preferred embodiments, the hybrid microRNA or precursor thereof comprises at least one or more of the following 5′ SR binding sites: SRSF1-(GTGAGGA), SRSF6-(TGAGGA), or SRSF5-(ATAAAGG). In preferred embodiments, the hybrid microRNA or precursor thereof comprises at least one or more of the following 3′ SR binding sites: SRSF6-(TGCTTC), or SRSF2-(GCTTCCTA, AACCCGCG). In embodiments, the hybrid microRNA or precursor thereof comprises a 5′ sequence with at least 70%, 75%, 80%, 85%, 90%, 91%, 92%, 93%, 94%, 95%, 96%, 97%, 98%, 99% or 100% sequence identity to SEQ ID NO: 42 or SEQ ID NO: 44. In embodiments, the hybrid microRNA or precursor thereof comprises a 3′ sequence with at least 70%, 75%, 80%, 85%, 90%, 91%, 92%, 93%, 94%, 95%, 96%, 97%, 98%, 99% or 100% sequence identity to SEQ ID NO: 43 or 45.

Mature microRNAs associated with the RISC complex can induce degradation or inhibit translation of the target polynucleotide. In embodiments. the target polynucleotide is an mRNA. Whether a microRNA or hybrid microRNA will result in cleavage or inhibition of translation depends on the degree of complementarity between the microRNA and the target polynucleotide. For microRNAs that are fully complementary to the target polynucleotide sequence, the target mRNA will be degraded by the RISC. For microRNAs that are only partially complementary to the target polynucleotide sequence, the RISC will instead inhibit translation of the target polynucleotide. In embodiments, inhibiting translation may occur by preventing ribosome binding, translational repression, or deadenylation (Ambros, V. (2001) microRNAs: tiny regulators with great potential. Cell 107(7):823-6; Buckingham, S. (2003) The major world of microRNAs, Horizon Symposia: Understanding the RNAissance. Nature Publishing Group, Nature, 1-3; He et al. (2009) Let-7a elevates p21 WAF1 levels by targeting of NIRF and suppresses the growth of A549 lung cancer cells. FEBS Letters 583:3501-3507). In animals, including humans, translational inhibition of target polynucleotides is the preferred mechanism of post-transcriptional regulation by endogenous micro RNAs, whereas most microRNA-targeted mRNAs are degraded in plants. Therefore, the hybrid microRNA may “reduce”, “supress”, “decrease” or “inhibit” translation of the target polynucleotide. In embodiments, the hybrid microRNA may result in “degradation” of target mRNA by deadenylation, decapping, and/or recruitment of exonucleases and/or endonucleases. In embodiments, the hybrid microRNA may result in “cleavage” of the target polynucleotide by the Argonaute protein in the RISC (Xu et al. (2016). MicroRNA-mediated target mRNA cleavage and 3′ uridylation in human cells. Scientific Reports, 6: 30242). In embodiments, the inhibition of translation, or cleavage, of the target polynucleotide may be statistically significant. In embodiments, the inhibition of translation, or cleavage, of the target polynucleotide may result in a reduction of 100%, 95%, 90%, 85%, 80%, 75%, 70%, 65%, 60%, 55%, 50%, 45%, 40%, 35%, 30%, 25%, 20%, 15% 10% or 5% of the target polynucleotide. Therefore, in embodiments, the present invention also relates to a method of degrading a target polynucleotide or reducing or inhibiting translation of a target polynucleotide using one or more hybrid microRNAs or precursors thereof, or a polynucleotide encoding the same, vector, expression cassette, complex or composition. In embodiments, the present invention relates to the use of one or more hybrid microRNAs or precursors thereof, or a polynucleotide encoding the same, vector, expression cassette, complex or composition for use in degrading a target polynucleotide or reducing or inhibiting translation of a target polynucleotide.

Either the passenger strand, or mature microRNA strand is loaded into the RISC. If the passenger strand is preferentially loaded into the RISC, there may be a higher occurrence of off-target effects. Therefore, in embodiments, the hybrid microRNA or precursor thereof preferentially loads the guide strand into the RISC. In embodiments, the hybrid microRNA or precursor thereof results in preferential expression of the guide strand over the passenger strand. Methods of quantifying levels of guide or passenger strands, or of the target mRNA in general, may be determined using standard techniques in the art (Wang et al. (2007). Analysis of microRNA effector functions in vitro. Methods, 43(2):91-104), such as quantitative PCR (qPCR), RNA solution hybridization, nuclease protection, Northern hybridization, reverse transcription-polymerase chain reaction (RT-PCR), microarray, antibody binding, luciferase assay, enzyme linked immunosorbent assay (ELISA), Western blotting, radioimmunoassay (RIA), other immunoassays, and fluorescence activated cell analysis (FACS). Also, the consequences of target polynucleotide suppression can be confirmed by examination of the phenotype of the cell or organism. In embodiments, the hybrid microRNA may result in fewer off target effects compared to a commercial control vector, or other microRNA. Off target effects may be determined by, for example, the detection of side effects such as the inhibition of expression of non-target genes and consequent phenotypic changes.

The present invention also relates to methods producing a hybrid microRNA or precursor thereof that suppresses expression of a target mRNA. Such methods may use a hybrid microRNA backbone as described herein, into which a guide sequence specific for a target mRNA is cloned. For instance, the method may comprise: (i) selecting, preparing or obtaining a hybrid microRNA backbone sequence, or a polynucleotide, expression cassette or vector that encodes said hybrid microRNA backbone sequence; wherein the hybrid microRNA backbone sequence comprises a stem sequence from a first intronic microRNA, and a loop sequence from a second microRNA; and (ii) cloning a guide sequence that binds specifically to a target mRNA into said hybrid microRNA backbone sequence, or into said polynucleotide, expression cassette or vector; wherein the stem, loop and guide sequences together form a hybrid hairpin that is processed to form a mature microRNA comprising the guide sequence, and the mature microRNA directs degradation of and/or inhibits translation of said target mRNA.

### Vectors

In embodiments, the hybrid microRNA or precursor thereof may be encoded by a polynucleotide. The present disclosure also encompasses multiplexed applications, e.g. where multiple different stem, loop and/or guide sequences are used together (e.g. expressed via a single transcript). For instance, in some embodiments the polynucleotide may encode two or more hybrid microRNAs or precursors thereof. In embodiments, the polynucleotide may encode two or more hybrid microRNAs or precursors thereof with the same stem and loop sequences, but different guide sequences, e.g. two or more guide sequences directed to different parts of a target mRNA. In embodiments, the polynucleotide may encode two or more hybrid microRNAs or precursor thereof with the same guide sequences but different stem and loop sequences. In embodiments, the hybrid microRNA or precursor thereof may be encoded by DNA.

In preferred embodiments, the polynucleotide encoding the hybrid microRNA or precursor thereof may be in a plasmid, construct, expression cassette or vector. In embodiments, the polynucleotide encoding the hybrid microRNA or precursor thereof may comprise, consist, or consist essentially of at least 10, 15, 20, 30, 50 or 100 nucleotide residues, or the full sequence of SEQ ID NOs: 2, 7, 14-17, 19-20, 49-54 or 58-75. In embodiments, the polynucleotide encoding the hybrid microRNA or precursor thereof may have at least 60%, 65%, 70%, 75%, 80%, 82%, 85%, 87%, 90%, 91%, 92%, 93%, 94%, 95%, 96%, 97%, 98%, 99% or 100% homology (e.g. sequence identity) across at least 8, 9, 10, 11, 12, 13, 14, 15, 20, 30, 50 or 100 base pairs, or across the full sequence of SEQ ID NOs: 2, 7, 14-17, 19-20, 49-54, or 58-75.

As used herein, the term “vector” refers to any means that is capable of delivery and optionally expressing any of the polynucleotides or hybrid microRNA or precursors thereof as described herein to a host cell, patient or subject. Examples of vectors include “plasmids” (also referred to as “expression constructs”), “RNA expression vectors”, expression cassette, nucleic acids complexed with a delivery vehicle such as liposome, peptide or poloxamer, viral vectors (including retroviral vectors, adenovirus vectors, poxvirus vectors, lentiviral vectors, herpesvirus vectors or adeno-associated virus vectors), yeast artificial chromosome, or phage (bacteria) vectors. As used herein, the terms “plasmids”, “construct” or “vector” may refer to single-stranded, double-stranded and/or circular or linearised. In preferred embodiments, the term “pMiX” or “plasmid miRNA-expression” refer to a plasmid comprising a polynucleotide encoding the hybrid microRNA or precursor thereof of the present disclosure. In embodiments, polypeptides encoding two or more different hybrid microRNAs or precursors thereof same are carried on the same vector. In embodiments, polypeptides encoding different hybrid microRNAs or precursors thereof are carried on different vectors.

In some embodiments, a cloning platform is envisaged where the vector may only comprise a stem sequence, and loop sequence, but not the final guide sequence. This allows the end user to clone their desired guide sequence into the vector. Such vectors lacking the guide sequence are referred to as hybrid microRNA “backbones”. In embodiments, the hybrid microRNA “backbones” may further comprise one or more elements for maintenance of the vector, such as an origin of replication (ORI) sequence, bacterial selectable marker genes or other nucleotide sequences that are not required for expression of the hybrid microRNA or precursor thereof.

In embodiments, there is provided isolated or substantially purified polynucleotides essentially free from components that normally accompany or interact with the polynucleotide as found in its naturally occurring environment. Thus, an isolated or purified polynucleotide is substantially free of other cellular material, or culture medium when produced by recombinant techniques, or substantially free of chemical precursors or other chemicals when chemically synthesized.

In embodiments, the polynucleotide sequence encoding the primary microRNA may be operably linked to regulatory sequences that control the expression of the transcript, such as an enhancer or promoter.

As used herein, “operably linked” is intended to mean a functional linkage between two or more elements. Operably linked elements may be contiguous or non-contiguous. In embodiments, the polynucleotide encoding one or more hybrid microRNAs or precursors thereof is operably linked to a “regulatory sequence” or “regulatory region” such as a promoter, transcriptional regulatory region or translational termination region.

As used herein, the term ‘promoter’ refers to a nucleic acid that serves to control the transcription of one or more polynucleotides, located upstream from the polynucleotide(s) sequence. In some embodiments, the promoter sequence is expressed in many tissue/cell types (i.e., ubiquitous), while in other embodiments, the promoter is tissue or cell specific. In preferred embodiments, the promoter sequence is specific for neuronal cells. In some embodiments the promoter may be constitutive or inducible. Non-limiting examples of ubiquitous promoters include CMV, CAG, Ube, human beta-actin, Ubc, SV40 or EF1a. Non-limiting examples of neuron-specific promoters include neuron-specific enolase (NSE), Synapsin, calcium/calmodulin-dependent protein kinase II, tubulin alpha I, and MECPs. In other embodiments, the promoter sequence is specific for muscle cells, such as muscle creatine kinase (MCK). Non-limiting examples of promoters suitable for use in plasmid vectors to drive expression of guides, pre-gRNA or mature gRNA include RNA polymerase III promoters. Examples of RNA polymerase III promoters include U6 and H1. Accordingly, an “enhancer” is a polynucleotide, which can stimulate promoter activity, and may be an innate element of the promoter or a heterologous element inserted to enhance the level or tissue-specificity of a promoter.

In some embodiments, the hybrid microRNA or precursor thereof, or polynucleotide encoding the same is associated with, or comprises, a detectable agent, such as a reporter agent or detectable epitope tags. Suitable reporter agents include, but are not limited to: proteins that mediate antibiotic resistance (e.g., ampicillin resistance, neomycin resistance, G418 resistance, or puromycin resistance), coloured, fluorescent or luminescent proteins (e.g., a green fluorescent protein (GFP), an enhanced GFP (eGFP), a blue fluorescent protein or its derivatives (EBFP, EBFP2, Azurite, mKalamal), a cyan fluorescent protein or its derivatives (ECFP, Cerulean, CyPet, mTurquoise2), a yellow fluorescent protein and its derivatives (YFP, Citrine, Venus, YPet), UnaG, dsRed, eqFP61 1, Dronpa, TagRFPs, KFP, EosFP, Dendra, IrisFP, mcherry, or luciferase), or proteins which mediate enhanced cell growth and/or gene amplification (e.g., dihydrofolate reductase). Suitable epitope tags may include one or more copies of the FLAG™, polyhistidine (His), myc, tandem affinity purification (TAP), or hemagglutinin (HA) tags or any detectable amino acid sequence.

In embodiments, the polynucleotide sequence encoding the hybrid microRNA, hairpin or stem-loop structure or precursor thereof is located downstream of (i.e. 3′ to) a pre-mRNA 3′end cleavage site or polyadenylation (“polyA”) signal sequence. In embodiments, the polynucleotide encoding the hybrid microRNA, hairpin or stem-loop structure or precursor thereof is located between 85-1500 nucleotides (or “base pairs” or “bp”), preferably, between 250-1000 nucleotides 3′ to a pre-mRNA 3′ cleavage site or polyadenylation signal sequence. In embodiments, the pre-mRNA 3′ cleavage site is determined by a position of a polyadenylation signal sequence. In embodiments, the pre-mRNA 3′ cleavage site is 3′ to the polyadenylation signal sequence. In embodiments, the polyadenylation signal sequence comprises AAUAAA, AGUAAA, UAUAAA, CAUAAA, GAUAAA, AAUAUA, AAUACA, AAUAGA, AACUAAA, AAGAAA, or AAUGAA (SEQ ID NOs: 55-57, and 76-83) (Tian and Graber (2012). Signals for pre-mRNA cleavage and polyadenylation. Wiley Interdisciplinary Reviews RNA, 3(3): 385-396.). In embodiments, the cleavage site is located 10-30 nucleotides (or “base pairs”) downstream of the polyadenylation signal, preferably 20-30 nucleotides (or “base pairs”).

As used herein, the term “expression cassette” refers to DNA consisting of a polynucleotide encoding a sequence of interest, and a regulatory sequence. In embodiments, the term “expression cassette” refers to DNA comprising, consisting or consisting essentially of one or more polynucleotides encoding a hybrid microRNA or precursor thereof and a regulatory sequence.

Viral vectors may be either replication competent or replication defective vectors. In embodiments, the vector is an adeno-associated virus (AAV), preferably, the vector is a recombinant AAV (rAAV). AAV belongs to the genus Dependoparvovirus within the family Parvoviridae. The AAV life cycle is dependent on the presence of a helper virus, such as adeno viruses. AAVs are composed of an icosahedral protein capsid ˜26 nm in diameter and a single-stranded DNA genome of ˜4.7 kb which is flanked by inverted terminal repeats (ITRs) that are required for genome replication and packaging. rAAVs are composed of the same capsid sequence and structure as found in wild-type AAVs. However, rAAVs encapsidate genomes that are devoid of all wild type AAV protein-coding sequences which are instead replaced with therapeutic gene expression cassettes (also referred to as a transgene). The only sequences of viral origin in rAAVs are the ITRs, which are needed to guide genome replication and packaging during vector production. The complete removal of viral coding sequences maximizes the packaging capacity of rAAVs and contributes to their low immunogenicity and cytotoxicity when delivered in vivo. Therefore, as used herein, the term ‘AAV vector’ refers to a vector comprising one or more polynucleotides of interest (or transgenes) that are flanked by AAV ITR sequences.

There are several identified AAV serotypes, and different serotypes interact with serum proteins in different ways. Serology of AAVs is an important functional characteristic for cell specific transduction efficiency within the CNS. In embodiments the AAV serotype is: AAV1, AAV2, AAV3, AAV5, AAV6, AAV7, AAV8 and AAV9. AAV hybrid serotypes or pseudo-serotypes have been created by viral engineering, which are constructed with integrated genome containing (cis-acting) inverted terminal repeats (ITR) of AAV2 and capsid genes of other serotypes for increased viral specificity and transduction. Therefore, in embodiments, the AAV vector is a hybrid serotype. In embodiments the AAV is AAV-PHP.B, —PHP.eB or PHP.S. Whereas AAV-PHP.B transduces the majority of neurons and astrocytes across many regions of the central nervous system, AAV-PHP.eB has been found to reduce the required viral load.

In embodiments, the AAV may be self-complementary and/or single stranded. Self-complementary cassette genomes are single stranded inverted repeat sequences with a mutated ITR in the middle of the molecule that folds to form double stranded DNA. Deletion of a terminal resolution site (trs) from the central ITR means that the ITR can no longer act as a replication origin but is still capable of forming a hairpin structure Thus, upon uncoating, sense and antisense strands anneal by folding together at the hairpin to form transcriptionally active dimers (McCarty et al, 2001 & 2003). The maximum DNA sequence packaged in self-complimentary cassette is 2.3 kb and 4.7 kb for single-stranded ITR cassettes. In embodiments, the AAV comprises a neutral “stuffing sequence” to enable efficient AAV encapsulation. In embodiments, the neutral stuffing sequence is between 0.5-2.5 kilobase pairs in length, preferably between 1.0-2.0 kilo base pairs, even more preferably around 1.5 kilo base pairs. In embodiments, “vMix” is used to refer to a self-complementary AAV comprising a polynucleotide encoding one or more hybrid microRNA.

In embodiments, the virus may be a lentivirus. Various lentiviruses are known in the art, such as the HIV based vectors described in U.S. Pat. Nos. 6,143,520, or lentiviruses described in U.S. Pat. No. 6,013,516, which are incorporated by reference. In embodiments, the lentivirus may be a first, second or third generation lentivirus. In preferred embodiments, the lentivirus may be a third generation lentivirus, as described in Milone and O'Doherty. (2018). Clinical use of lentiviral vectors. Leukemia, 32: 1529-1541.

The hybrid microRNA, precursor thereof or polynucleotide encoding the same can be administered together with lipid molecules such as cationic lipids, or peptides, or in the context of polymeric scaffolds which can facilitate their delivery. In embodiments, the hybrid microRNA, precursor thereof or polynucleotide encoding the same may be administered using a commercial lipid transfection kit such as Lipofectamine™ (Thermo Fisher Scientific). The hybrid microRNA, precursor thereof or polynucleotide encoding the same may be administered using a lipidic molecule, such as a “liposome”. The term “liposome”, “lipid vesicle” or “vesicle” are used interchangeably to refer to an aqueous compartment enclosed by a lipid bilayer refers to a vesicle composed of at least one bilayer of amphipathic molecules which form a membrane separating an intravesicular medium from an external medium. The intravesicular medium constitutes the internal aqueous core of the liposome. Hydrophilic molecules or components, can be encapsulated inside the internal aqueous core of the liposome via active methods of encapsulation known in the art or methods described in US 2021/0186894, herein incorporated by reference. Briefly, the liposome may be prepared by reverse phase evaporation, ethanol injection, heating, lipid film hydration. Loading of the hybrid microRNA, precursor thereof, or polynucleotide encoding the same may be via passive or active loading techniques. In embodiments, passive loading techniques such as mechanical dispersion (such as sonication, French pressure cell, freeze-thawing, lipid film hydration, micro-emulsification, membrane extrusion or drying and reconstituting), solvent dispersion or detergent removal may be used (also see Akbarzadeh et al. (2013) Liposome: classification, preparation and applications. Nanoscale Research Letters, 8(1): 102). Hydrophobic molecules or components can be entrapped inside the membrane. In embodiments, the liposome may be a multilamellar vesicle (MLV), small unilamellar liposome vesicle (SUV), large unilamellar vesicle (LUV) or cochleate vesicles. In embodiments, the delivery composition may be a micelle or other lipid-based delivery vehicle, such as those described in Torchilin et al. Advanced Drug Delivery Reviews, 58(14):1532-55 (2006).

As used herein, the term “transduction” refers to the process by which a sequence of foreign nucleotides is introduced into the cell by a virus.

As used herein, the term “transfection” refers to the introduction of DNA into the recipient eukaryotic cells.

As used herein, the terms “composition” or “pharmaceutical composition” are used interchangeably and refer to any composition comprising one or more hybrid microRNAs or precursors thereof, or a polynucleotide encoding the same, vector, expression cassette or complex as described herein. By “pharmaceutical composition” it is generally meant that the compositions is suitable for pharmaceutical use, e.g. in mammals such as humans. Thus a pharmaceutical composition is typically in a form that is suitable for administration (e.g. to humans) via oral or parenteral administration (e.g. intravenous or subcutaneous administration). Thus a pharmaceutical composition may exclude the presence of components not suitable for administration (e.g. to humans). Particularly in the case of pharmaceutical compositions for injection (e.g. intravenous or subcutaneous administration), it is preferred that the composition is sterile.

In embodiments, the composition may further comprise a pharmaceutically acceptable carrier, diluents or excipients. As used herein the term “pharmaceutically acceptable carrier, diluent or excipient” is intended to include sterile solvents or powders, dispersion media, coatings, antibacterial and antifungal agents, disintegrating agents, lubricants, glidant, sweeting or flavouring agents, antioxidants, buffers, chelating agents, binding agents, isotonic and absorption delaying agents, or suitable mixtures thereof. Preferably, the diluent or carrier is sterile water, saline solution, fixed oils, polyethylene glycols, glycerine, propylene glycol, bacteriostatic water, phosphate-buffered saline (PBS), Cermophor EL™ (BASF, Parsippany, N.J), other solvents or suitable mixtures thereof. Preferably, the antibacterial or antifungal agents include benzyl alcohol, parabens, chlorobutanol, phenol, ascorbic acid, thimerosal, methyl parabens, or suitable mixtures thereof. In embodiments, the antioxidants include ascorbic acid or sodium bisulphite of suitable mixtures thereof. In embodiments, the chelating agents include ethylenediaminetetraacetic acid (EDTA). In embodiments, the absorption delaying agents include aluminium monostearate or gelatin, or suitable mixtures thereof. In embodiments, isotonic agents include sugars, mannitol, sorbitol, or sodium chloride, or suitable mixtures thereof. In embodiments, the binding agent is microcrystalline cellulose, gum tragacanth, or gelatin, or suitable mixtures thereof. In embodiments, the excipient may be starch or lactose, or suitable mixtures thereof. In embodiments, the disintegrating agent may be alginic acid, Primogel or corn starch, or suitable mixtures thereof. In embodiments, the lubricant may be magnesium stearate or sterotes, or suitable mixtures thereof. In embodiments, the glidant may be colloidal silicon dioxide. In embodiments, the sweetening or flavouring agents may be sucrose, saccharin, peppermint, methyl salicylate or orange flavouring.

In embodiments, the present invention relates to a kit for cloning a guide sequence into a hybrid microRNA backbone. In embodiments the kit may comprise a polynucleotide encoding one or more hybrid microRNA “backbones” (i.e., without the final guide sequence) or precursors thereof, and one or more reagents suitable for cloning a guide sequence that binds specifically to a target mRNA into said polynucleotide. In embodiments, the polynucleotide encoding one or more hybrid microRNAs or precursors thereof in the kit may be on a vector. The kit may further comprise oligonucleotides, primers or probes for cloning the guide into the polynucleotide, for example, by PCR. In some embodiments, a kit may comprise instructions for one or more of the methods disclosed.

### Treatment or Prevention

In embodiments, the hybrid microRNA, precursors thereof, polynucleotide encoding the same, vector, expression cassette, complex or composition may be for use in the treatment or prevention of a disease, disorder or condition. The present disclosure also relates to methods of treating or preventing a disease, disorder or condition using the hybrid microRNA, precursors thereof, polynucleotide encoding the same, vector, expression cassette, complex or composition as described herein.

The terms “disease”, “disorder”, “condition”, “illness” or “pathology” are used interchangeably herein to refer to any condition that is or is predicted to be associated with symptoms or underlying pathology. In general, any disease or disorder can be treated using the hybrid microRNA of the present disclosure by selecting a suitable guide sequence, e.g. a guide sequence that targets a gene associated with the disease.

In one embodiment, the disease to be treated is a neurodegenerative disease. For instance, one or more hybrid microRNA, precursors thereof, a polynucleotide encoding the same, vector, expression cassette, complex or composition as described herein may be used to treat a neurodegenerative disease. Neurodegenerative diseases are characterized by the loss of specific neurons, and are complex, progressive, disabling, and often fatal. Neurodegenerative diseases can be divided into acute and chronic neurodegenerative diseases. In embodiments, the neurodegenerative disease may be chronic. In preferred embodiments, the neurodegenerative disease may be Amyotrophic Lateral Sclerosis (ALS), Spinocerebellar ataxia type 2 (SCA2), Parkinson's disease (PD), Huntington's Disease (HD), Alzheimer's disease (AD), Supranuclear Palsy, and Frontotemporal Dementia (FTD) (Xu et al., 2021). In embodiments the disease may be Duchenne Muscular Dystrophy (DMD). In embodiments, the disease may be associated with a genetic mutation. In preferred embodiments the disease is associated with a mutation of Progranulin (PGRN), Huntington (HTT), Ataxin 2 (ATXN2), Superoxidase dismutase 1 (SOD1), chromosome 9 open reading frame (C9orf72), Fused in Sarcoma (FUS), microtubule associated protein tau (MAPT), leucine-rich repeat kinase 2 (LRKK2), alpha-synuclein (SCNA), dystrophin (DMD)).

The terms “treating” or “treatment” as used herein refer to reducing the severity and/or frequency of symptoms, reducing the underlying pathological markers, eliminating symptoms and/or pathology, arresting the development or progression of symptoms and/or pathology, slowing the progression of symptoms and/or pathology, eliminating the symptoms and/or pathology, or improving or ameliorating pathology/damage already caused by the disease, condition or disorder.

The terms “preventing” or “prophylaxis” as used herein refer to the prevention of the occurrence of symptoms and/or pathology, delaying the onset of symptoms and/or pathology. Therefore, “preventing” or “prophylaxis” in particular, applies when a patient or subject has a disease-causing mutation(s) in their genes, or is at risk of developing a disease but does not yet display symptoms or identifiable pathology.

As used herein, the terms “subject”, “patient” or “individual” are used interchangeably and refer to vertebrates, preferably mammals such as human patients and non-human primates, as well as other animals such as bovine, equine, canine, ovine, feline, murine and the like. In preferred embodiments, the subject, patient or individual is human. Accordingly, the term “subject” or “patient” as used herein means any mammalian patient or subject diagnosed with, predisposed to, or suspected of having a specified disease. Identification of diseases may be established through standard clinical tests or assessments, such as genetic testing.

As used herein, the terms “administering”, “administer” or “administration” means providing to a subject or patient one or more hybrid microRNAs, precursors thereof, polynucleotide encoding the same, vector, expression cassette, complex or composition as described herein using any method of delivery known to those skilled in the art to treat or prevent a disease, disorder or condition in a patient or subject. In embodiments, routes of delivery of the one or more hybrid microRNA, precursors thereof, a polynucleotide encoding the same, vector, expression cassette, complex or composition as described herein include intravenous, intradermal, subcutaneous, intraperitoneal, intramuscular, intrathecal or direct injection into the main site of the disease, e.g. the brain, inhalation, rectally (suppository or retention enema), vaginally, orally (capsules, tablets, solutions or troches), transmucosal or transdermal (topical e.g., skin patches, opthalamic, intranasal) application. In alternative embodiment, the one or more hybrid microRNAs, precursors thereof, polynucleotide encoding the same, vector, expression cassette, complex or composition as described herein is delivered directly to the cerebrospinal fluid (CSF), or brain, by a route of administration such as intrastriatal (IS), or intracerebroventricular (ICV) administration. The one or more hybrid microRNAs, precursors thereof, polynucleotide encoding the same, vector, expression cassette, complex or composition as described herein can also be administered by any method suitable for administration of nucleic acid agents. These methods include gene guns, bio-injectors, and needle-free methods such as the mammalian transdermal needle-free vaccination with powder-form vaccine as disclosed in U.S. Pat. No. 6,168,587. If desired to facilitate repeated or frequent infusions, implantation of a delivery device, e.g., a pump, semi-permanent stent (e g., intravenous, intraperitoneal, intracisternal or intracapsular), or reservoir may be used. In embodiments encompassing inhalation, the one or more hybrid microRNAs, precursors thereof, polynucleotide encoding the same, vector, expression cassette, complex or composition as described herein are delivered in the form of an aerosol spray from a pressured container or dispenser which contains a suitable propellant or nebuliser. In embodiments the propellant may be a gas such as carbon dioxide.

As used herein, the term “therapeutically effective amount” or “therapeutically effective dose” refers to an amount of the one or more hybrid microRNAs, precursors thereof, polynucleotide encoding the same, vector, expression cassette, complex or composition as described herein that, when administered to a patient or subject with a disease, is sufficient to cause a qualitative or quantitative reduction in the severity or frequency of symptoms of that disease, disorder or condition, and/or a reduction in the underlying pathological markers or mechanisms. In addition, a “therapeutically effective amount” also refers to an amount of one or more one or more hybrid microRNAs, precursors thereof, polynucleotide encoding the same, vector, expression cassette, complex or composition as described herein that, when administered to a patient or subject with a predicted or diagnosed disease but without symptoms, is sufficient to cause a qualitative or quantitative reduction in the underlying pathology markers or mechanisms, or prevent onset.

In a preferred embodiment, the therapeutically effective amount of one or more hybrid microRNAs, precursors thereof, polynucleotide encoding the same, vector, expression cassette, complex or composition as described herein may be administered only once. Preferably, the therapeutically effective amount of one or more hybrid microRNAs, precursors thereof, polynucleotide encoding the same, vector, expression cassette, complex or composition as described herein of the present invention is administered multiple times. In one embodiment, a patient or subject is administered an initial dose, and one or more maintenance doses. Certain factors may influence the dosage required to effectively treat a subject or patient, including but not limited to the severity of the disease, disorder or condition, previous or concurrent treatments, the general health and/or age of the subject, and other diseases present. It will also be appreciated that the effective dosage of the one or more hybrid microRNAs, precursors thereof, polynucleotide encoding the same, vector, expression cassette, complex or composition as described herein for treatment may increase or decrease over the course of a particular treatment.

In an alternative embodiment, the therapeutically effective dose may be administered with other therapies or methods (a “secondary therapy”). Example secondary therapies can be to alleviate symptoms, be neuroprotective, or restorative. Such methods and compositions may be modified for use in the present invention where appropriate.

The invention will now be described by way of example only, with reference to the following non-limiting embodiments.

### EXAMPLES

### Example 1: Custom MiRNA Hairpin Design and Production

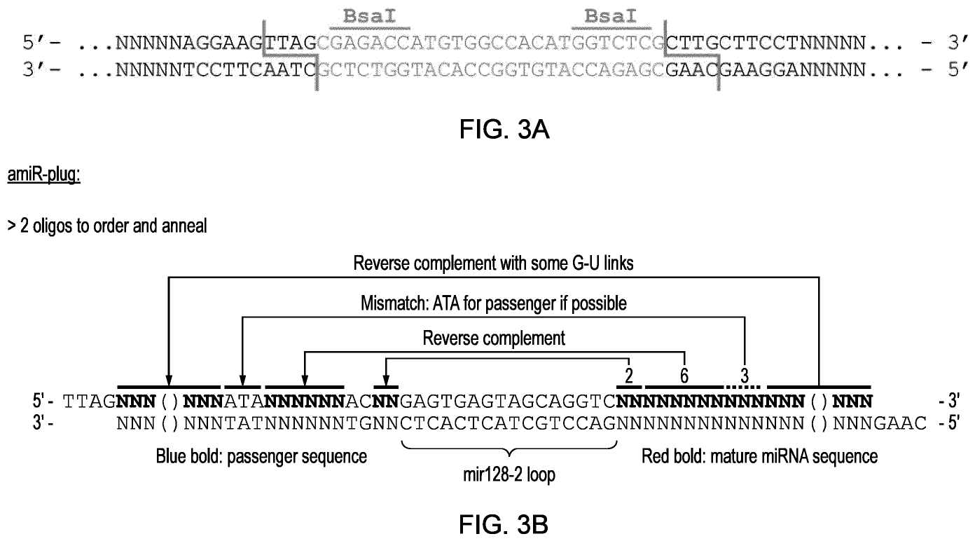

To create a plasmid coding miRNA against the target sequence, the miRNA hairpin (or pre-miRNA) is ligated into an empty pMiX backbone vector (FIG. 3; SEQ ID NO: 1).

The hairpin is generated by annealing two single-stranded oligonucleotides to create a small double-stranded DNA. The hairpin includes:
a) The guide or mature miRNA DNA sequence (5′ to 3′) or reverse complement of the target sequence.
b) The terminal loop GAGTGAGTAGCAGGTC (SEQ ID NO: 2).
c) The passenger sequence designed as following:
   i. Use reverse complement of the guide sequence (5′ to 3′) and number nucleotides from the 3′ end.
   ii. Add AC (SEQ ID NO: 3) between the 2^{nd} and 3^{rd} nucleotide.
   iii. Replace nucleotides 9, 10 and 11 by ATA (SEQ ID NO: 4).
   iv. To generate G-U pairs at RNA level, replace from the 14^{th} nucleotide with up to 3 A or C by respectively G or T, avoiding two substitutions in a row.
d) The hairpin is finally assembled in the following order (from 5′ to 3′): passenger, loop, and guide sequences.

The first single-stranded oligonucleotide is equivalent to the top strand of the designed hairpin, with TTAG (SEQ ID NO: 5) at the 5′. The second single-stranded oligonucleotide is the bottom strand of the designed hairpin, with CAAG (SEQ ID NO: 6) at the 5′.

The two single-stranded oligos (100 μM) were then annealed at 95° C. for 4 minutes in a dry block heater, using 2 μL of each oligo in 100 μL of DNase/RNase free water. Then, the heat block was removed from the heater and left on the bench to cool at room temperature for 1 hour. The mixture was then transferred to 4° C. for 5 minutes before dilution in water at a ratio of 1:15. Ligation into the pMiX backbone vector (SEQ ID NO: 1) was then performed with NEB Golden Gate Assembly kit (New England Biolabs, E1601) following the manufacturer instructions. To do this, 1 μL of diluted oligos was mixed with 1 μL of empty pMiX plasmid (75 ng/μL), 1 μL of Golden Gate Enzyme mix, 1X T4 DNA ligase buffer, and water up to 20 μL. The reaction is incubated for 30 minutes at 37° C., 5 minutes at 60° C., and kept at 4° C. until proceeding to competent cell (Top10 E. coli (ThermoFisher Scientific, C404006) transformation. To transform Top10 E. coli, 2 μL of the ligated plasmid mix was added to 25 μL of cells and incubated on ice for thirty minutes before heat shock in a 42° C. water bath for 30 seconds and placing back on ice for 2 minutes. 100 μL of S.O.C. medium was then added to the cells and incubated at 37° C. for 30 minutes with agitation. Bacteria were plated on agar (Sigma-Aldrich, L2897) plates supplemented with 50 μg/mL carbenicillin (Sigma-Aldrich, C1389) and incubated at 37° C. overnight. Colonies were then picked and amplified in 4 mL LB broth (Sigma-Aldrich, L3152) supplemented with 50 μg/mL carbenicillin overnight at 37° C. before DNA extraction using a miniprep kit (QIAGEN). To identify bacteria with the ligated plasmid, restriction analysis using EcoRV, PmlI and MscI for 30 minutes at 37° C. before running on a 1.5% Agarose gel.

### Example 2: Cell Culture Methods

### Cell Culture

Human kidney cell line (HEK-293, ECACC, 85120602) and Human cervix epitheloid carcinoma cell line (HeLa, ECACC, 930210130 were maintained at 37° C. in a humidified chamber with 5% CO₂ and cultured in Dulbecco's Modified Medium (DMEM, Gibco, 31966-021) supplied with 10% heat inactivated Fetal Bovine Serum (FBS, Gibco, 10500-064) and 10 U/mL Penicillin-Streptomycin (Gibco, 15140-122). Mouse muscle myoblast cell line (c2c12, ECACC, 91031101) were cultured DMEM supplied with 20% FBS and 10 U/mL Penicillin-Streptomycin. The cells were passaged every third day using TrypLE (Gibco, 12604-021) and plated at a 10⁶ cells in 150 cm² flask. Embryonic day 18 (E18) Sprague Dawley Rat neurons were cultured at 37° C., 5% CO₂ in Neurobasal medium (1X) (Gibco, 21103-049) supplemented with B27 (50X) (Gibco, 17504-044), 1% Penicillin Streptomycin (Gibco, 15140-122) and 1% L-glutamine (Life Technologies, 25030081).

### Transfection of Cells

HEK-293, HeLa and c2c12 cells were plated one day before transfection in 24-well plates (50,000 cells per well). The transfection of HEK-293, HeLa and rat cortical neurons was performed using Lipofectamine 2000 (ThermoFisher Scientific, 11668-019) and Opti-MEM I Reduced Serum Medium (Gibco, 31985-047) as following: 1 μL of lipofectamine, 100 μL of Opti-MEM and a maximum of 500 ng of DNA per well. The transfection mix was incubated 30 minutes at room temperature before adding to cells. The c2c12 cells were transfected with 80 μL of DMEM, 2 μL of 1 mg/mL polyethylenimine (PEI, Polyscience, 24885) and 400 ng of DNA. To knockdown Drosha in HEK-293 cells, 2 pmol of siRNAs (SMARTpool: ONTARGETplus, Horizon Discovery, L-016996-00-0005) were transfected into the cells using 1 μL of Lipofectamine 2000 and 100 μL of Opti-MEM per well.

### Dual Luciferase Reporter Assay (DLR)

HEK293 cells were transfected with pMiX and DLR reporter plasmids (pmir-GLO, Promega, E1330) at a 50:1 ratio as described above. After 24 hours, cells were washed with PBS and lysed with 200 μL of passive lysis buffer from DLR assay kit (Promega, E1910). The DLR assay was carried out with 20 μL of lysate in a white 96-well plate and a plate luminometer prepared according to the following protocol: (1) set injector 1 to dispense 100 μL Luciferase Assay Reagent II, (2) for measurements, use a 2-second delay and a 10-second read for firefly luciferase activity, (3) set injector 2 to dispense 100 μL Stop & GLO Reagent, followed by a 2-second delay and 10-second read time for Renilla luciferase activity.

To test knockdown efficiency when targeting a coding sequence (CDS), a miRNA targeting the coding sequence of Firefly luciferase (Fluc1, SEQ ID: 94) was designed using BLOCK-iT RNAi Designer software (https://rnaidesigner.thermofisher.com/rnaiexpress/) and cloned in pcDNA6.2 according to manufacturer instruction (ThermoFisher Scientific, K493500). This miRNA was then adapted to pMiX vectors according to Example 1. To test knockdown efficiency when targeting 5′UTR and 3′UTR, an endogenous miRNA (mir-128, SEQ ID: 95) was cloned in pcDNA6.2 and pMiX vectors. Two different DLR reporters were generated with the full match target sequence of mir-128 either before (5′UTR) or after (3′UTR) the coding sequence of Firefly luciferase.

### RT-PCR and RT-qPCR

Total RNA was extracted from cells using NEB Total RNA kit (New England Biolabs, T2010) and cDNA was synthesized with 1 μg of total RNA using LunaScript RT Supermix (NEB, E3010).

To check splicing of pMiX1-3 vector, a PCR was performed using 7.5 μL of OneTaq Hot Start Quick-Load 2X Master Mix (NEB, M0488), 0.5 μL of 10 μM primers and 1 μL of cDNA diluted 1:100, in a total volume of 15 μL. The cycling conditions were: (1) 2 minutes at 95° C., (2) 35 cycles with 10 seconds at 95° C., 10 seconds at 55° C. and 1 minute at 72° C., (3) 5 minutes at 72° C. PCRs were loaded in a 1.5% agarose gel. The primers used are: pMiX1 (AGTCCCAAGCTGGCTAG (SEQ ID NO: 96); CCGGACACGCTGAACT (SEQ ID NO: 97)), pMiX2 (CAGCACGACTTCTTCAAGTC (SEQ ID NO: 98); TGTACTCCAGCTTGTGCC (SEQ ID NO: 99)), and pMiX3 (CGATCACATGGTCCTGCT (SEQ ID NO: 100); CACACAGAAAACAGCTATGACC (SEQ ID NO: 101)).

For gene expression, qPCR were performed on QuantStudio 7 (Applied Biosystem) and Luna Universal qPCR Master Mix (NEB, M3003) following a standard protocol. Briefly, in a total of 20 μL, 2 μL of cDNA diluted at 1:200 were used with 0.5 μL of 10 μM oligos and 10 μL of Master Mix. The cycling conditions were: (1) 1 minute at 95° C., (2) 40 cycles with 15 seconds at 95° C. and 30 seconds at 60° C., (3) a melting curve.

The expression of miRNA was measured using miScript II RT Kit (Qiagen, 218161) and QuantiTect SYBR Green PCR Kit (Qiagen, 204143). The qPCR was performed in 20 μL with 2 μL of cDNA diluted at 1:10, 1 μL of 10 μM Universal primer, 1 μL of 10 μM miRNA specific primer, 10 μL of Master Mix. The cycling conditions were: (1) 15 minute at 95° C., (2) 40 cycles with 15 seconds at 94° C., 30 seconds at 55° C. and 30 seconds at 70° C., (3) a melting curve.

The primers used are: Firefly (GTGGTGTGCAGCGAGAATAG (SEQ ID NO: 102); CGCTCGTTGTAGATGTCGTTAG (SEQ ID NO: 103)), Renilla (GAGAAGGGCGAGGTTAGACG (SEQ ID NO: 104); TGGAAAAGAACCCAGGGTCG (SEQ ID NO: 105)). GAPDH (GTCTCCTCTGACTTCAACAGCG (SEQ ID NO: 106); ACCACCCTGTTGCTGTAGCCAA (SEQ ID NO: 107)), Drosha (TAGGCTGTGGGAAAGGACCAAG (SEQ ID NO: 108); GTTCGATGAACCGCTTCTGATG (SEQ ID NO: 109)), mir-128 (TCACAGTGAACCGGTCTC (SEQ ID NO: 110)), pMiX passenger of mir-128 (AGGGGGGCCGATACACT (SEQ ID NO: 111)), pcDNA6.2 passenger of mir-128 (AAAGAGACGTTCACTGTG (SEQ ID NO: 112)), mir-Fluc1 (TATTCTCGCTGCACACCAC (SEQ ID NO: 113)), pMiX passenger of mir-Fluc1 (TTGTGGTGTGATACGAGAAAC (SEQ ID NO: 114)), pcDNA6.2 passenger of mir-Fluc1 (CGTGGTGCAGCGAGAATA (SEQ ID NO: 68)).

### Western Blotting

Total protein was extracted from cells by washing the cells with PBS before adding 150 μL of Protein lysis buffer (prepared by placing 1 protease inhibitor tablet (ThermoFisher Scientific, 04693159001) into 10 mL NP-40 lysis buffer (10 mM Tris-HCl pH 7.5; 150 mM NaCl; 0.5 mM EDTA; 0.5% NP-40). The wells were then scraped with a pipette tip and transferred to microtubes before incubation for 30 minutes on ice, with vortexing every 5 minutes. Samples were then centrifuged at 10,000×g for 10 minutes at 4° C. and the supernatant stored at −20° C. for use. Prior to the Western blot, the total protein in the samples was quantified using the Bio-Rad DC Protein Assay (Bio-Rad, 500-0112) following the manufacturer protocol. Around 10 μg sample was supplemented with loading buffer consisting of 2X Sodium dodecyl sulfate buffer (SDS; 106 mM Tris-HCl, 106 mM Tris-Base, 0.74 mM SDS (Sigma-Aldrich), 0.5 mM EDTA, 1 M glycerol, 0.2 mM Brilliant Blue (Sigma-Aldrich), 0.175 mM Phenol Red (Sigma-Aldrich)) and 50 mM dithiothreitol (DTT; Invitrogen) to a total volume of 15 μL, and incubated for 10 minutes at 95° C. Samples were loaded, along with 2 μL Precision Plus Protein™ Dual Xtra Prestained Protein Standards (Bio-Rad, 1610377) onto pre-cast Novex NuPAGE 10% Bis-Tris 26-well Midi Gels (ThermoFisher Scientific, WG1203BOX) with Invitrogen Novex NuPAGE MOPS SDS Running Buffer (ThermoFisher Scientific, NP000102) and run at constant 100V for 45 minutes. Gels were then soaked in NuPAGE Transfer Buffer (ThermoFisher Scientific, NP00061) briefly before transferring the protein using the iBlot™ 2 Gel Transfer Device (ThermoFisher Scientific, IB21001) for 20V for 1 min, 23V for 4 min, then 25V for 2 min. Western Blocking Reagent (10%; Roche, 34608600) dissolved in PBS (1X; Sigma-Aldrich, P4417-100TAB) was used to block the protein membranes for one hour at room temperature. Afterwards, the membrane was incubated in primary antibody (1:1000 anti-PGRN (Abcam, ab191211) and 1:10,000 anti-GAPDH (Abcam, ab82485)) diluted in 5% blocking buffer overnight at 4° C. After washing three times with TBS-T (TBS: VWR, K859-200TABS; 0.1% Tween-20: Sigma-Aldrich, P9416-100ML) membranes were incubated with secondary antibodies (goat anti-mouse IgG (H+L) DyLight 680/800 conjugate (ThermoFisher Scientific, SA535521, 1:5,000) and goat anti-rabbit (H+L) DyLight 680/800 conjugate (ThermoFisher Scientific, SA535571, 1:5,000)) for one hour at room temperature. After three washes (2 with TBS-T, 1 with TBS) membranes were scanned on Odyssey CLx infrared imaging system (Li-Cor Biosciences). The intensities of each band of proteins was measured using ImageJ and ImageStudio-light.

### Example 3: Self-Complement AAV Virus Production and Purification

For each virus, ten 145 cm² plates of low passage (≤P30) HEK-293 cells at approximately 80% confluence were used. For transfection, 800 μL of 1 mg/mL polyethylenimine (PEI; Polyscience) was added to 15mL of serum free DMEM. In a separate tube, 240 μg of Adeno helper plasmid containing essential genes from the adenoviral genome that support rescue and replication of AAV genomes (Aldevron, pALD-X80) 80 μg of Rep2/Cap9 plasmid (de novo synthesized Rep2/Cap9 plasmid (Gao et al., 2004. Journal of Virology, 78: 6381-6388) and 80 μg of the single stranded transgene plasmid was added to serum free DMEM to a final volume of 30 mL. The mixture containing DNA was then filtered into the PEI-containing mixture through a 0.45 μm polyethersulfone (PES, Sartorius, Epsom, UK) syringe filter. The solution was mixed and incubated at room temperature for 15 minutes. The solution was then added drop wise to each 145 cm² dish and cells then incubated at 37° C. for 72 hours. To harvest the AAV virus particles, 72 hours after transfection, the supernatant and transfected cell pellets were collected and subjected to freeze thaw cycles. Freeze thaw cycles were performed by placing the supernatant and pellets into liquid nitrogen and then into a 37° C. water bath for five cycles. All supernatant and pellets were treated with DENARASE (50 Unit/mL, c-LEcta) at 37° C. for 30 minutes before centrifugation at 2000×g for 30 minutes at 18° C. The supernatants were then filtered with 0.22 μM pore size filter (Millipore) and applied to a pre-equilibrized AAVX POROS affinity column (Thermo Fisher Scientific) on the AKTA system (AKTA Pure, Cytiva) for AAV purification. The viral titre of purified AAV particles were determined using Q-PCR and analysis of the packaged viral genome performed using Alkaline gel electrophoresis. Endotoxin (Pierce™ LAL Chromogenic Endotoxin Quantitation Kit (Thermo Fisher) and mycoplasma (MycoAlert (Lonza)) levels were also tested for in vitro and in vivo application using kits and following the manufacturer protocols. The purified AAV was kept at −80° C.

### Example 4: Modifying Stem and Loop Sequences to Increase MiRNA Processing Efficiency

Transcript knockdown by short hairpin RNA (shRNA) is normally around 50%, however expression tracking, multiple target knockdown and tissue-specific expression is not possible with shRNA due to restricted promoter usage (Pol-III). To overcome these disadvantages, we developed hybrid microRNA constructs by reengineering sequences that originally come from endogenous miRNA and will allow us to use any type of cells, such as neuron- or glial-specific promoters (Pol-II). To do this, we used intronic miRNAs, which are relatively small in size (less then 1 kb) and abundant in the human brain (Table 1) (Panwar et al., 2017. Bioinformatics 33, btx019) for the stem and loop sequences. although the loop sequence may be any miRNA.

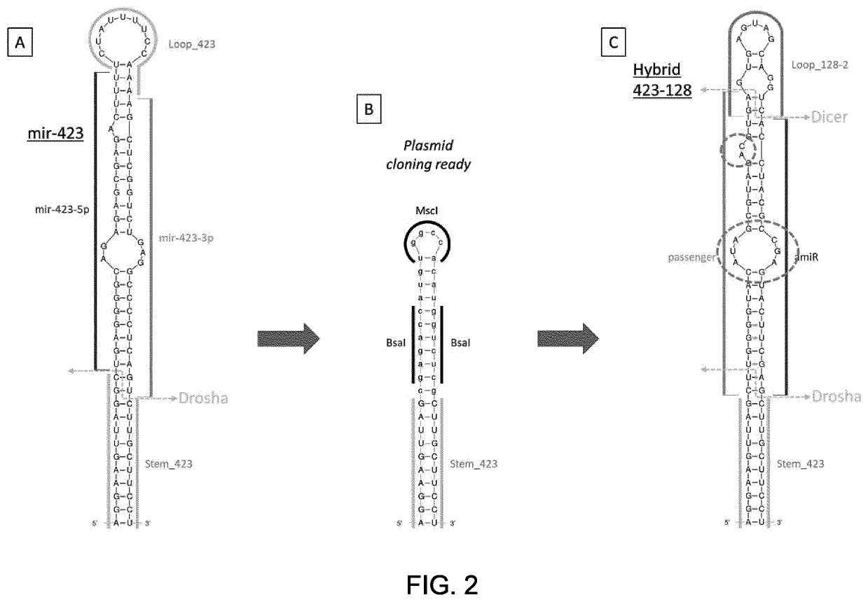

We selected miR-423 for the stem sequence, which harbours the Drosha cleavage site because it is highly expressed in the brain and located in a small 400 bp intron. We replaced its loop sequence with miRNA-128-2, chosen because it is also abundant in neurons and expresses only one strand of the hairpin as mature miRNA (Juhila et al., 2011. PloS One 6, e21495) (FIG. 2). We named this novel miRNA platform “pMiX “(plasmid miRNA-expression) and “vMiX” when it has been incorporated in a novel self-complimentary AAV viral expression Inverted Terminal Repeat (ITR) sequence.

To produce the pMiX, the methods outlined in Example 1 were followed. Briefly, the pre-mir-423 hairpin (above Drosha cleavage site) was removed using Q5 Site-Directed Mutagenesis Kit (NEB, E05540) and replaced by a cloning sequence (SEQ ID NO: 8) with two BsaI sites (type IIS restriction enzyme) to facilitate the cloning and a MscI site to screen bacteria after hairpin cloning (FIG. 2A. 2B. 3A). To insert the new hairpin carrying the custom artificial miRNA (amiR), a “plug in” construct was designed using two synthesised oligos and annealed to generate the double-stranded insert (FIG. 3B) as described in Example 1.

### Example 5: Developing an EGFP MiRNA Processing Reporter Using Intronic Sequences

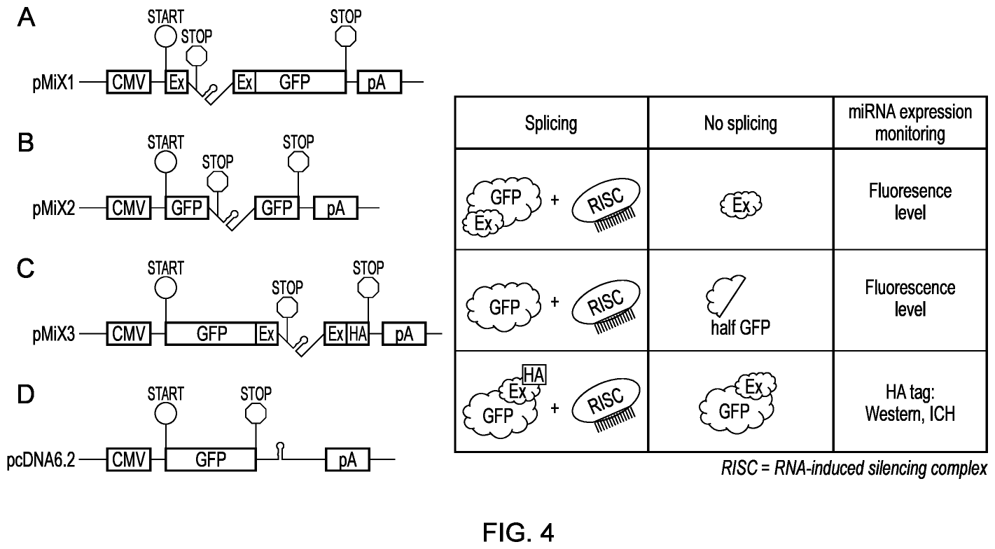

To test the efficacy of the pMiX system, we devised a series of constructs that incorporated the intronic miR-423-128 flanking sequence within the 5′ UTR (pMiX1; SEQ ID NO: 9), CDS (pMiX2; SEQ ID NO: 10), 3′UTR (pMiX3; SEQ ID NO: 11) of EGFP sequence (FIG. 4). The rate at which splicing occurs is reflected by the level of EGFP protein translation, quantified using a GFP quantification in microplates using a microplate reader.

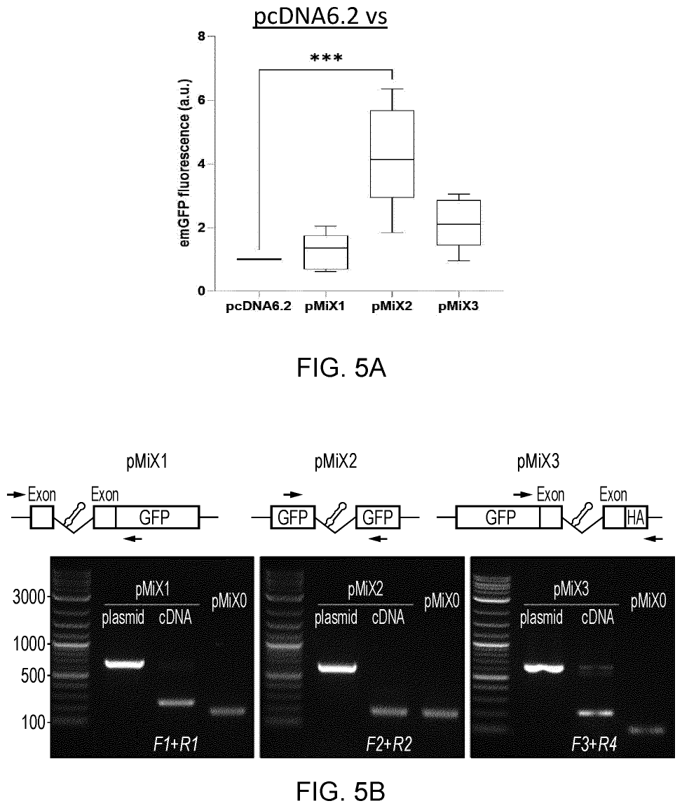

Therefore, to test the splicing of pMiX vectors, we measured GFP fluorescence of HEK-293 lysates (FIG. 5A). Splicing was compared to the commercially available, miRNA cloning system pCDNA 6.2-GW/EMGFP-miR (BLOCK-iTTM, Thermofisher). The pCDNA 6.2 constructs express artificial miRNAs engineered to have 100% homology to target sequence and result in target mRNA cleavage. However, this system is not very efficient when targeting the coding sequence (CDS) or the 5′ untranslated region (UTR) achieving on average 0-40% knock down. Therefore, a pCDNA 6.2 construct comprising an GFP-miR-155 sequence was used as the control for the present experiments. The fluorescence of pMiX2 was higher than other vectors. pMiX1 and 3 showed lower levels of fluorescence despite only differing in the position of the miR-423-128 sequence. Therefore, the position of the exons (5′ or 3′ of GFP) appear to have an influence on the GFP protein activity. Using RT-PCR (as described in Example 2), it was confirmed that only pMiX2 was fully spliced (FIG. 5B).

### Example 6: Knock Down Efficacy of PMiX MiRNA Constructs in a Dual Luciferase Assay

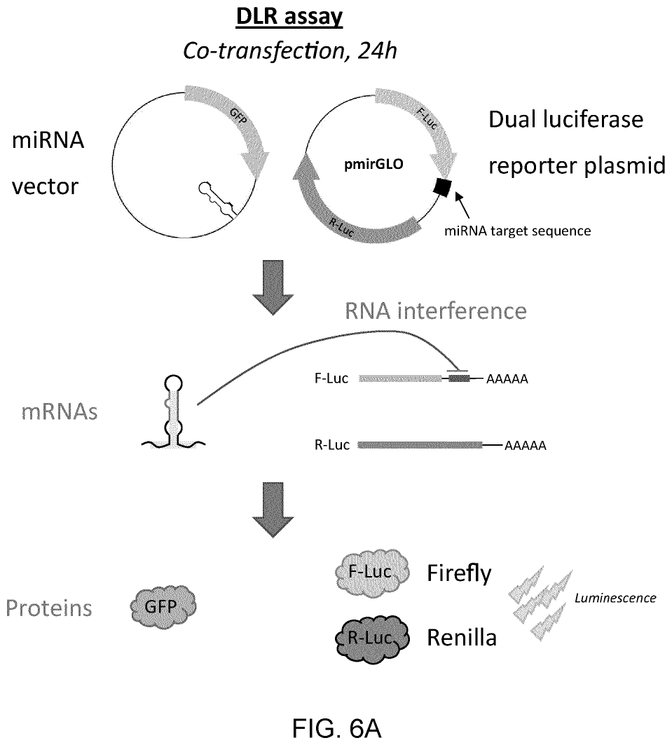

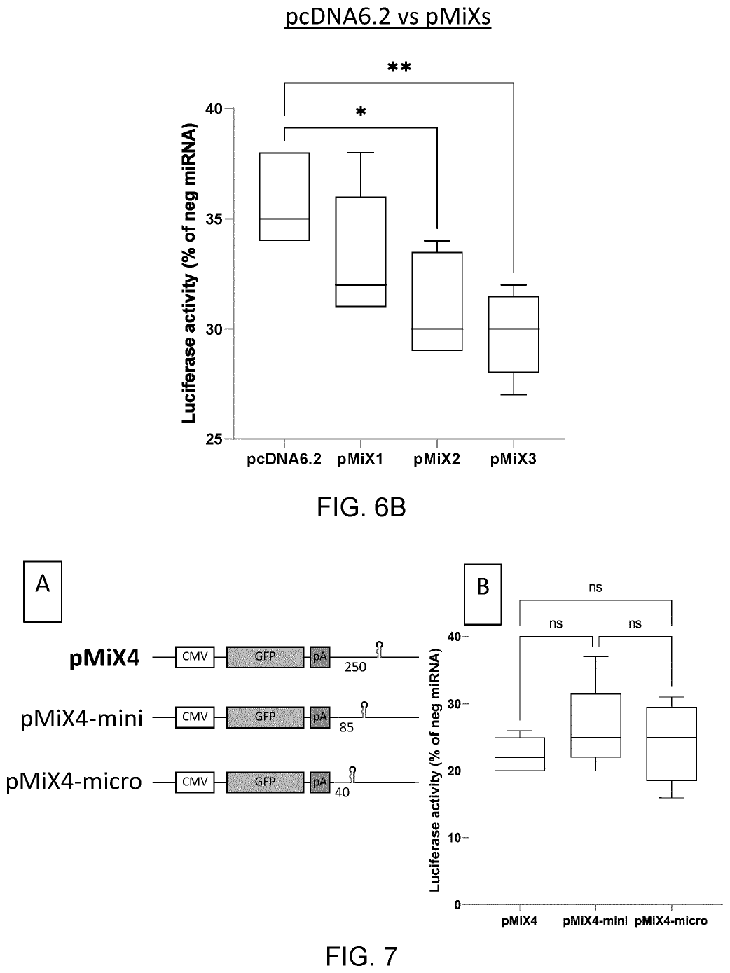

To test the efficacy of pMiX 1-3, we used a commercially available Dual Luciferase Reporter (DLR) plasmid (as described in Example 2) that has a miRNA target sequence within the 3′UTR of the Firefly luciferase gene. The DLR assay allowed us to measure the efficiency of miRNA knock-down by observing whether there was a decrease in the Firefly luciferase signal, which was normalized to the transfection efficiency by the Renilla luciferase signal (FIG. 6A). The knockdown efficiency of each construct was determined as pMiX1=66%, pMiX2=69%, and pMiX3=71%, which achieved only a modest increase in knockdown efficiency over the GFP-miR-155 construct pcDNA 6.2 at 64% knockdown (FIG. 6B).

### Example 7: Comparing Knock Down Efficacy Based on MiRNA Placement

In order to improve the processing efficiency of our hybrid miR-243-128 construct, we placed the construct after the polyA signalling sequence to determine whether processing by 3′ end cleavage increased the rate of miRNA generation (FIG. 7A). We placed our hybrid miRNA at varying distances from the 3′ end cleavage site (see FIG. 7A); 40 (pMiX4-micro; SEQ ID NO: 12), 85 (pMiX4-mini; SEQ ID NO: 13) and 250 (pMiX4; SEQ ID NO: 7) bp from the 3′ end cleavage site.

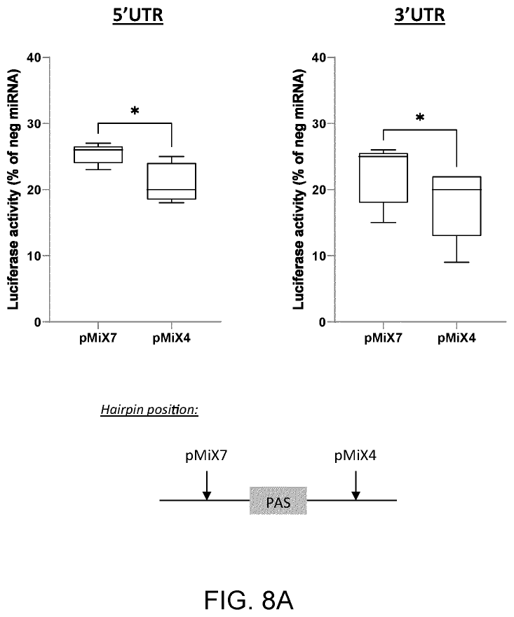

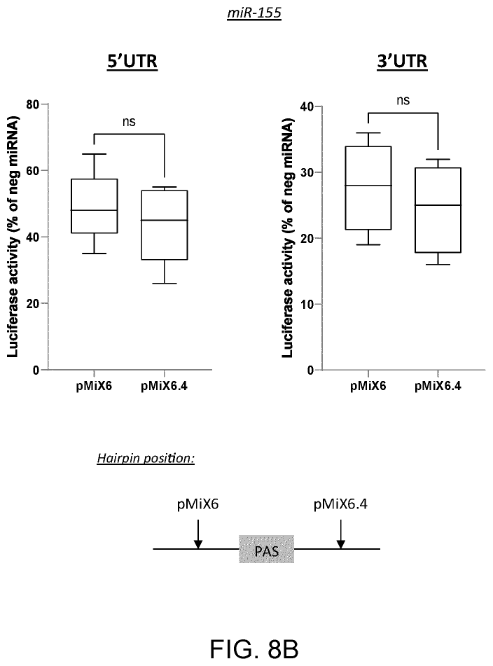

This further increased knock down with pMiX4 achieving 77% knockdown efficiency, whilst both pMix4-mini and pMix4-micro achieved a 73% knockdown efficacy (FIG. 7B). Compared to pMiX1-3, where the miR construct was placed before the polyA signalling sequence (see FIG. 4), placing the miRNA construct after the polyA dramatically enhanced the miRNA activity compared to intronic miRNA (pMiX2) (see FIGS. 6B and 7B). Similarly, when the intronic miR423 in the pMiX4 (after polyA) or pMiX7 system (before polyA) (FIG. 8A) showed significant reduction of the miRNA activity when the hairpin was placed before the polyA sequence (FIG. 8A).

To test whether the miRNA location before or after the polyA was the key element for the miRNA system, we tested positioning using a different miRNA. To do this, we used the miR-155 in pMiX6 (before polyA), and pMiX6.4 (after polyA) (see FIG. 8B). The activity was not significantly changed by the positioning for the miR-155 based system (pMiX6).

These results therefore suggest that the polyA enhancing miRNA activity was specific to the intronic sequence contained miR423.

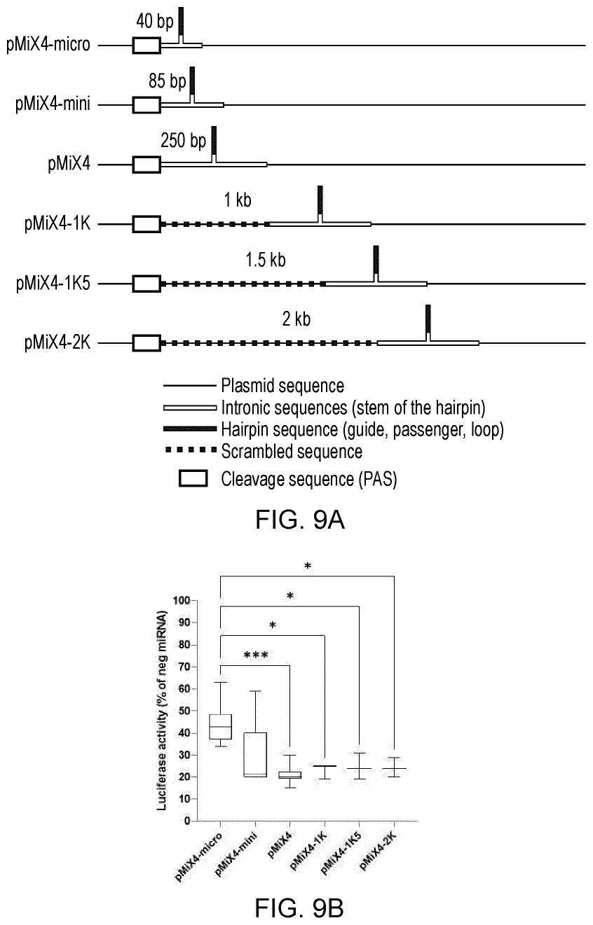

We then tested the effect of increasing the distance between the polyA signalling sequence and hairpin sequences by sequentially introducing sections of scramble sequence (FIG. 9A). As demonstrated in FIG. 9B, and in line with the experiments above, when the distance between the polyA signalling sequence to miR construct was reduced (in pMiX4-mini and pMix4-micro), the miRNA activity of pMiX4 was reduced. Increasing the distance between the polyA signalling sequence and hairpin results in reduced efficacy around 1.5 kb, and then complete inhibition after 2 kb (FIG. 9B). These results suggest that the distance between the poly A and hairpin sequences is optimally located between 80-1500 base pairs and diminished by shortening or lengthening.

### Example 8: Rate of Knockdown for Intronic and Pre-MRNA 3′ End Cleavage MiRNA

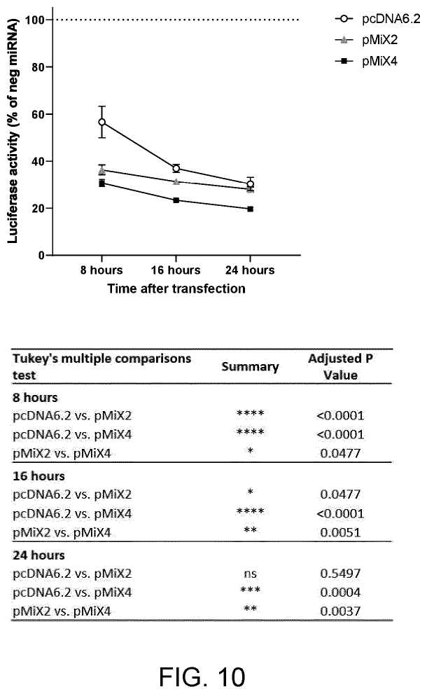

To check whether there were any rate limitations of the miRNA activity, we compared the efficiency of the luciferase signal knockdown in HEK293 cells at 8, 16 and 24 hours after transfection (see Example 2). As demonstrated in FIG. 10, pMiX2 and pMiX4 achieved more rapid and effective knock down than pcDNA6.2 at 8 and 16 hour time points, indicating greater efficiency in miRNA processing.

### Example 9: Testing Knockdown Efficiency of 5′UTR, CDS and 3′UTR Targets

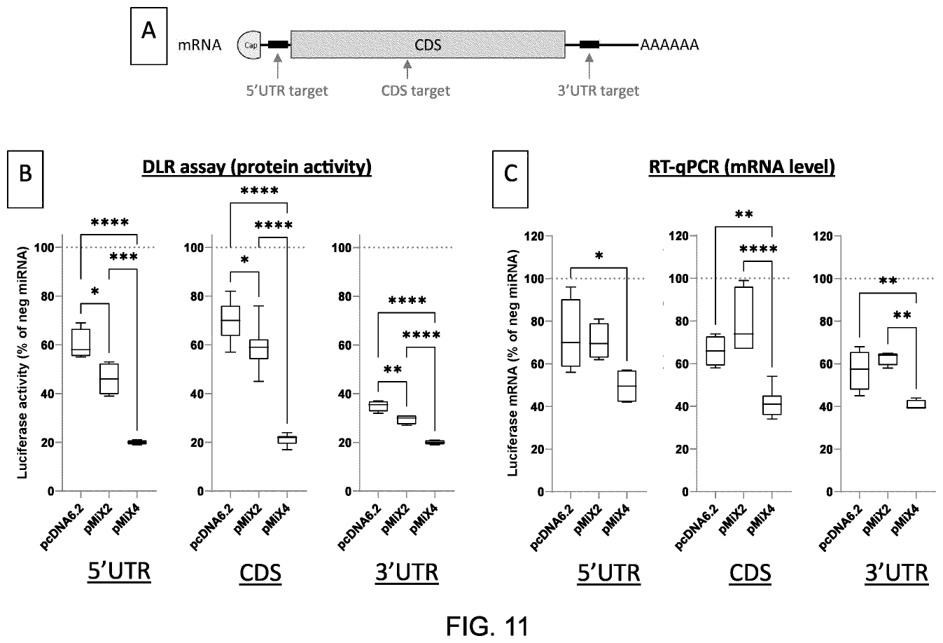

Conventionally, miRNA targets sequences within the 3′UTR, where it predominantly suppresses translation, but a small proportion of mRNAs are also degraded by the RISC complex. To test whether our pMix system was able to degrade target mRNA, we targeted sequences in the 5′UTR, coding sequence (CDS) and 3′ UTR of the luciferase gene for the DLR assay (FIG. 11A).

Measurements of luciferase activity showed that the pcDNA 6.2 was less effective at knocking down targets in the 5′UTR and CDS compared to pMiX2 and pMiX4 (FIG. 11B). pMix2, however, knocked down luciferase activity by 53% for 5′UTR, 42% for CDS and 70% for 3′UTR, while pMiX4 achieved 80% knock down for all three targets (FIG. 11B). The levels of luciferase mRNA were also quantified by qPCR and revealed that only the pMiX4 efficiently reduced the target mRNA (FIG. 11C). We therefore concluded that the novel pMiX4 construct was a highly efficient and effective miRNA platform to target any site within mRNA transcripts and substantially outperforms the leading commercial alternative (pcDNA6.2).

### Example 10: Comparison of MiRNA Processing to Generate Guide and Passenger Strands

The correct loading of either passenger or guide miRNA into the RNA induced Silencing Complex (RISC) is key for reducing off-target effects. Therefore, it is preferable to engineer an miRNA that preferentially loads the guide RNA, and not the passenger strand, into the RISC complex.

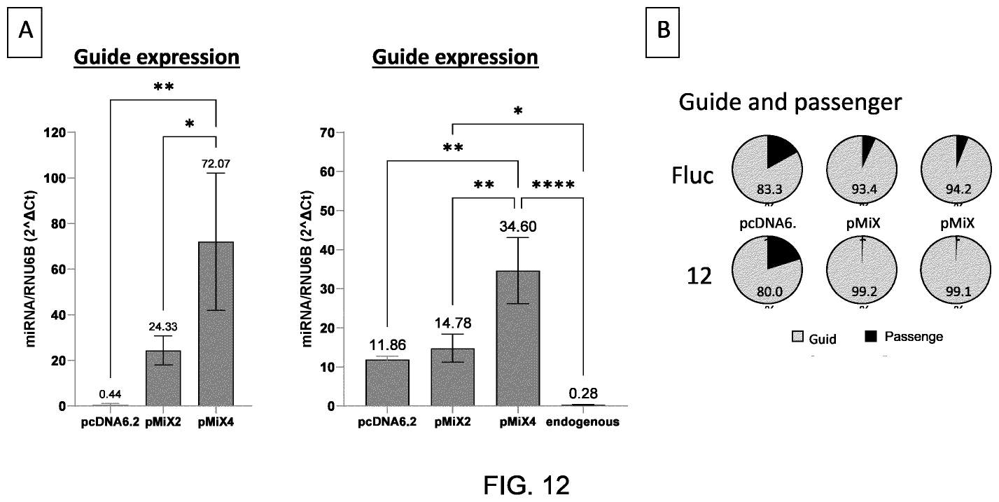

To test whether the hybrid miRNA-243-128 platform was able to preferentially load guide strands into RISC and leave passenger strands to be degraded, we quantified both guide and passenger strands by qPCR 24 hours after transfection with the hybrid miRNA construct. Hybrid miRNA in pMiX2 and pMiX4 vectors generated much higher levels of the guide than passenger strands for miRNA Fluc1 and miRNA 128, than pcDNA6.2 (FIG. 12A). pMiX2 and pMiX4 also produced much lower levels of passenger strand than the commercial vector pcDN6.2 for both miRNAs tested (FIG. 12B). Poor guide strand selectivity by the commercial vector has been previously reported (Maczuga et al., 2013. BMC Biotechnol, 2012. 12: p. 42) as 62% of miRNAs being from the passenger strand, when using the miR-155 flanking sequence in the pcDNA6.2 plasmid. Therefore. FIG. 12 demonstrates that our novel hybrid miRNA constructs are able to efficiently, and preferentially load the guide strand into the RISC complex, thereby achieving greater knock down with fewer off target effects.

### Example 11: Testing Drosha Dependence

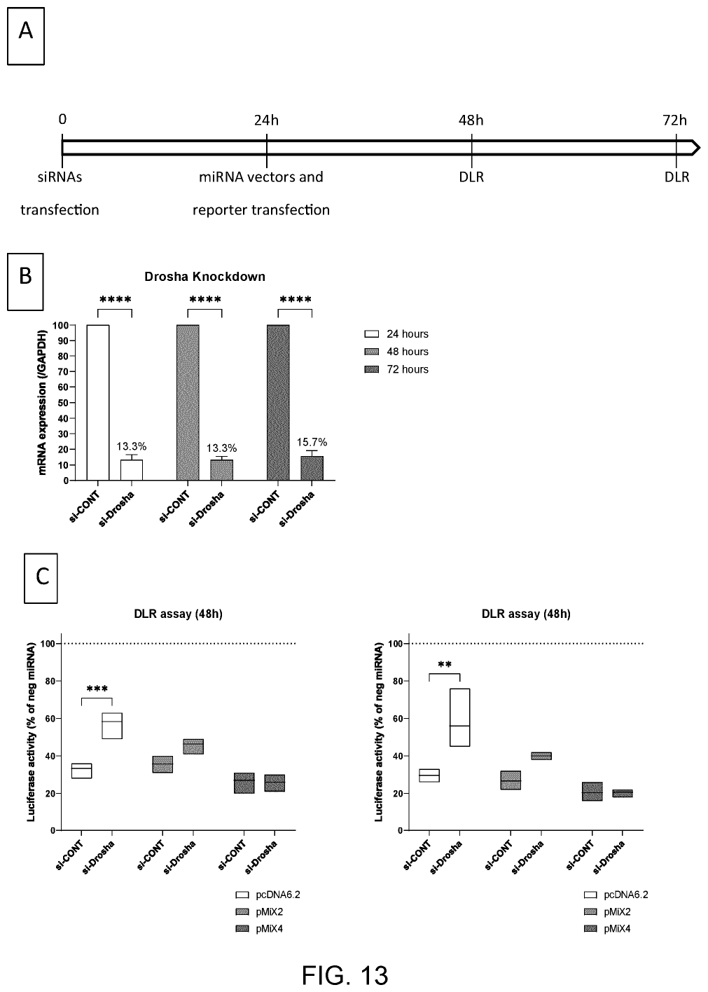

To assess whether the pMiX vectors are dependent on Drosha cleavage, we knocked-down Drosha in HEK 293 cells using siRNA (Dharmacon) and measured Drosha mRNA by qPCR. After 24 hours, cells were transfected with the DLR reporter plasmid and miRNA vectors expressing the 3′UTR of the luciferase (as described in Example 2) for 24 and 48 hours. After this time, DLR assays were performed (FIG. 13A). Knockdown of Drosha by siRNA left only 13.3%, 13.3% and 15.7% of Drosha mRNA detectable after 24, 48, and 72 hours respectively (FIG. 13B). Drosha knockdown in the HEK 293 cells reduced the silencing effect of the commercial vector (pcDNA6.2) by 50-75%, and the pMiX2 vector by 30-50% indicating a high degree of Drosha dependence on processing for both vectors. However, Drosha knockdown did not affect the efficiency of pMiX4 vector over the 48 hour period (FIG. 13C). These results therefore indicate that the processing of pMix4 miRNA could be Drosha independent.

### Example 12: Testing Silencing of Endogenous Genes Using the PMiX2 and PMiX4 Platform

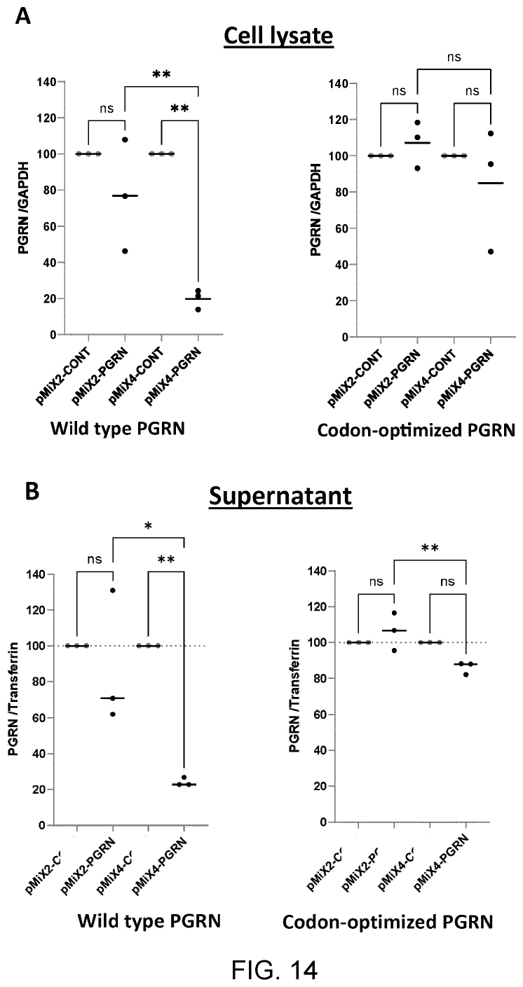

To test whether our miRNA-423-128 can efficiently knock down other coding mRNA, we designed a “plug in” guide sequence targeting 21 base pairs (bp) within the CDS sequence of the PGRN transcript, which encodes the secreted protein progranulin. In order to test the selectivity of the miRNA guide we compared the knock down of co-transfected wild-type protein (PGRN-WT) with a codon-optimised version (PGRN-CO), where the target sequence differs by only 4 base pairs. Scrambled guide sequence was used as a negative control. pMiX4 achieved excellent PGRN knock down in both the cell lysate and supernatant (75-80%) compared to pMix2 (20-40%) (FIG. 14A and 14B). Importantly, both pMiX2 and pMix4 did not reduce PGRN protein expression from codon-optimised PGRN plasmids, suggesting that the pMix system delivers highly sequence-specific knockdown.

### Example 13: Exploring PMiX4 MiRNA Enhancing Elements That Contribute to Its Efficacy

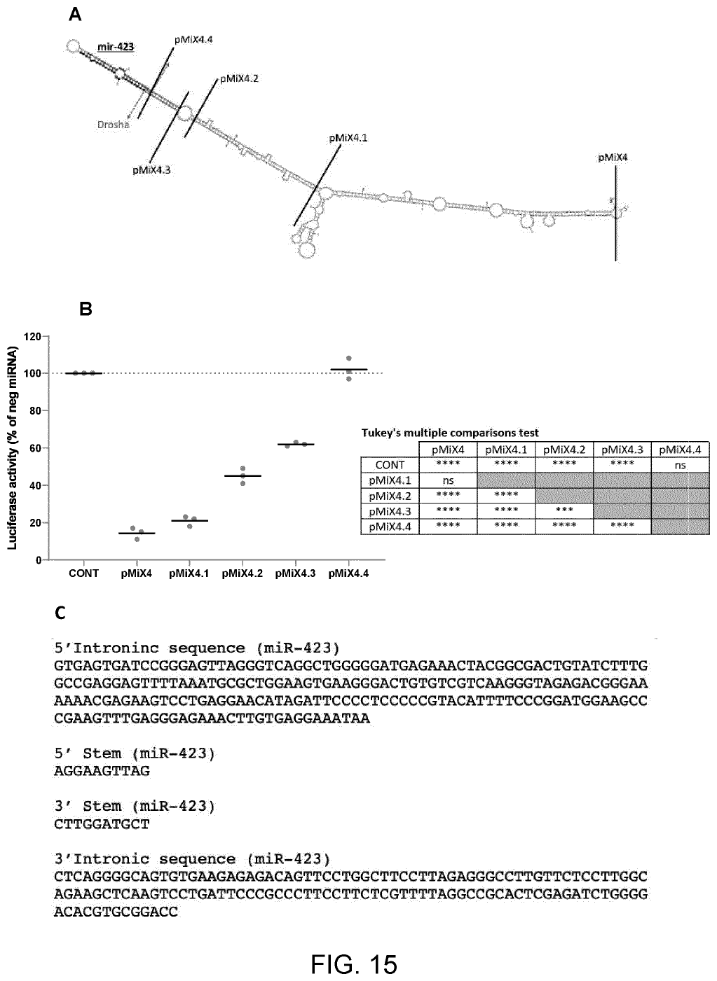

In order to determine the relative contribution of the adjacent intronic sequence and secondary structure to the efficacy of miR-423, we performed sequential deletions to create pMiX4.1 (SEQ ID NO: 14), pMiX4.2 (SEQ ID NO: 15), pMiX4.3 (SEQ ID NO: 16) and pMiX4.4 (SEQ ID NO: 17) according to big RNA Bulge sequence (FIG. 15A). As demonstrated in FIG. 15B, all of the intronic sequence and resulting secondary structure contributes to the processing efficiency and efficacy of miR-423 knockdown. The deletion of the intronic sequence of the miR-423 significantly inhibited the miRNA silencing activity (FIG. 15B) whilst the deletion between pMix4-pMix4.2 did not alter the activity. The results therefore suggest that the intervening sequence of pMix4-pMix4.2 can be spared. The intronic sequences of miR-243 are illustrated in FIG. 15C (SEQ ID NOs: 18-21).

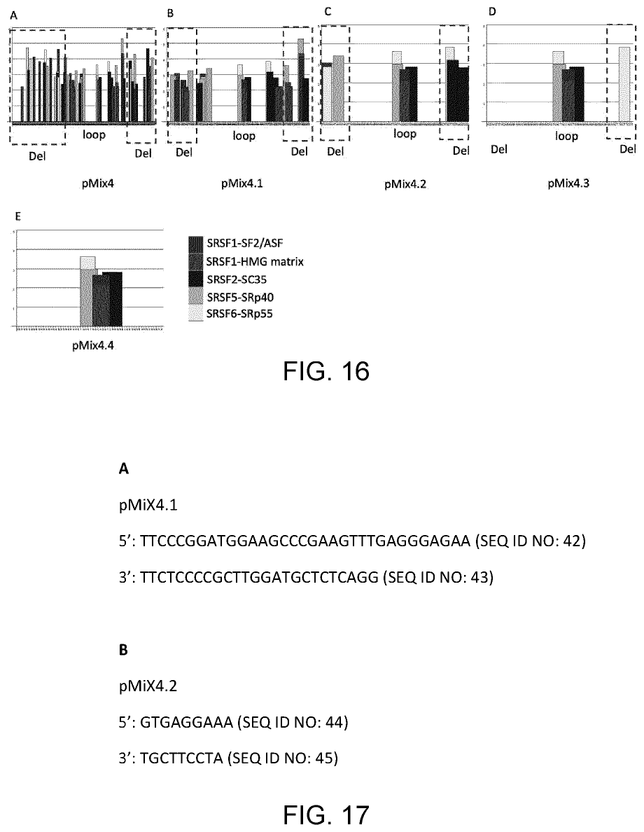

Mutations on exonic splicing enhancers (ESE) can cause exon skipping, which inhibits splicing activity. ESEs recruit Ser/Arg rich proteins (SR proteins) which enhance the splicing and maturation of mRNA in the nucleus. The first deletion of the of the pMiX4 was not dramatically altered the miRNA activity in pMix4.1. However, we observed significant inhibition of mRNA activity by further deletion from pMiX4.1 to 4.2 (FIG. 16). Suggesting the ESE sequence within these are could be the key enhancing element for the miRNA processing. The responsible 5′ sequence is SRSF5-(TTTCCCG (SEQ ID NO: 22); TTTGAGG (SEQ ID NO: 23)) SRSF1-(CGGATGG (SEQ ID NO: 24), AGCCCGA (SEQ ID NO: 25)), SRSF1-HMG(CGGATGG (SEQ ID NO: 26)), SRSF-(AACTTGTG (SEQ ID NO: 27)). The cover range of the sequence was between 33 bp. For the 3′ sequences were SRSF6-(TGAGTA (SEQ ID NO: 28)), SRSF5-(TTTCTCC (SEQ ID NO: 29), TCTCAGG (SEQ ID NO: 30)), SRSF1-HMG-(CTCCCCG (SEQ ID NO: 31), CCCCGCT (SEQ ID NO: 32), and CTCAGGG (SEQ ID NO: 33)), SRSF1-(CTCAGGG (SEQ ID NO: 34)), SRSF2-(GGGCAGTG (SEQ ID NO: 35)) which is around 25 bp (FIG. 16A-B).

The full sequence of SR binding site of pMIX4.1 and pMix4.2 are illustrated in FIG. 17 (SEQ ID NOs: 42-45).

### Example 14: Other Intronic Hybrid MiRNA Sequences

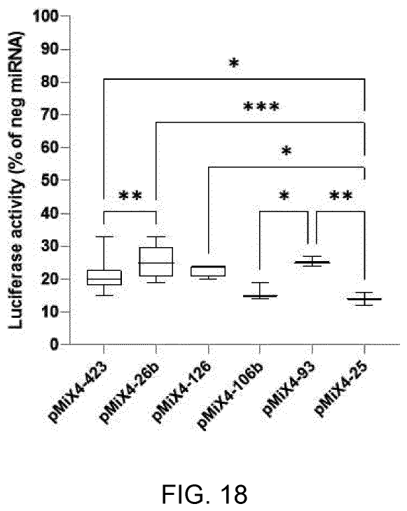

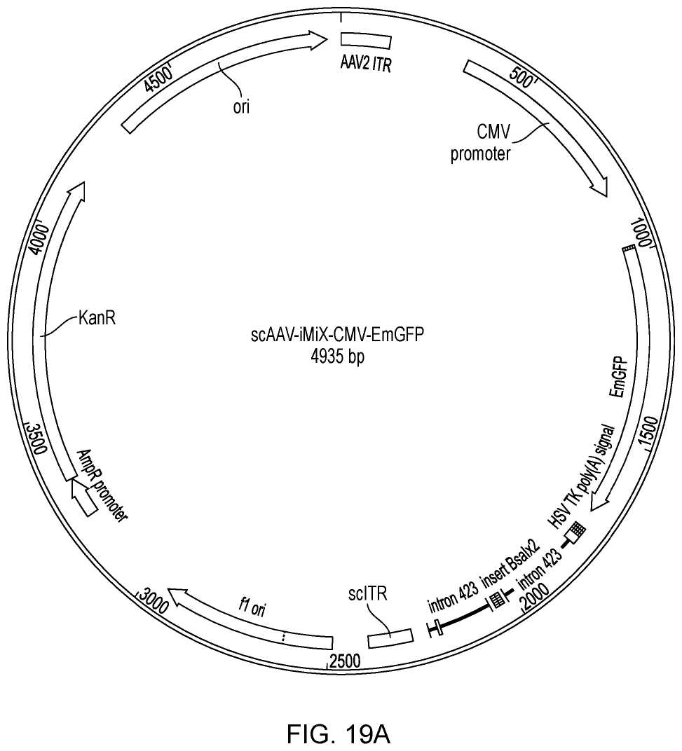

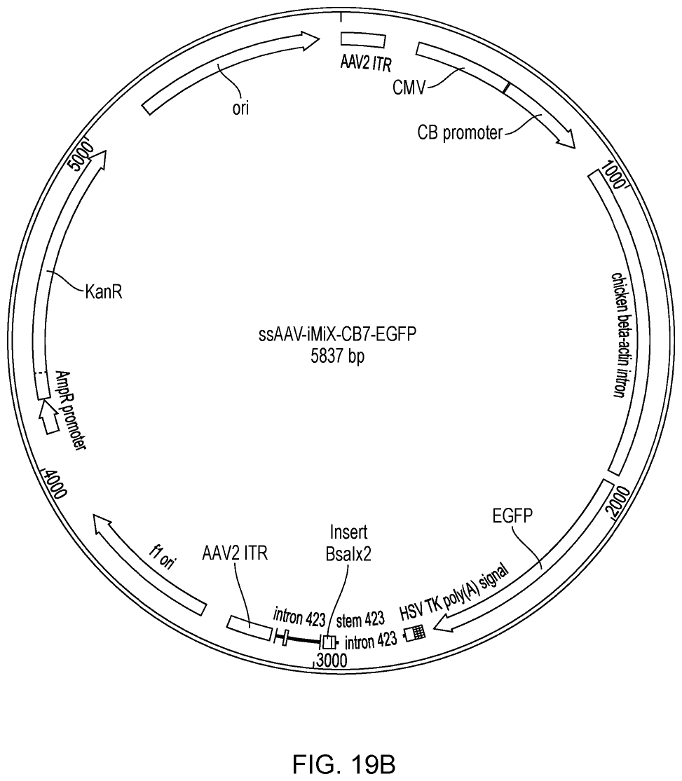

To test whether other intronic miRNA stem sequences could achieve similar levels of knockdown efficiency, we selected other brain-specific miRNAs from a public database (miRmine). The sequences of miR-26b, miR-126, miR-106b, miR-93, and miR-25, were hybridized with the miR-128 loop and tested for luciferase activity (as detailed in Example 2). As shown in FIG. 18, the alternative intronic miRNAs were able to deliver efficient knockdown efficient similar as miR-423.

### Example 15: Developing Optimal AAV Platform for MiRNA Cassette

The main limitation of using AAV is the limited genome capacity. Self-complementary cassette genomes are single stranded inverted repeat sequences with a mutated ITR in the middle of the molecule that folds to form double stranded DNA. Deletion of a terminal resolution site (trs) from the central ITR means that the ITR can no longer act as a replication origin but is still capable of forming a hairpin structure. Thus, upon uncoating, sense and antisense strands anneal by folding together at the hairpin to form transcriptionally active dimers (McCarty et al., 2001. Gene Therapy 8, 1248-1254 & McCarty et al., 2003. Gene therapy 10 (26), 2112-2118). The maximum DNA sequence packaged in self-complimentary cassette is 2.3 kb and 4.7 kb for single-stranded ITR cassettes. However, the location of miRNA hairpin in ITR shuttle could interfere with AAV replication and packaging, leading to poor capsidation and transduction. A major concern is that the proximity of the miRNA to the 3′ ITR will cause truncation of the transgene.

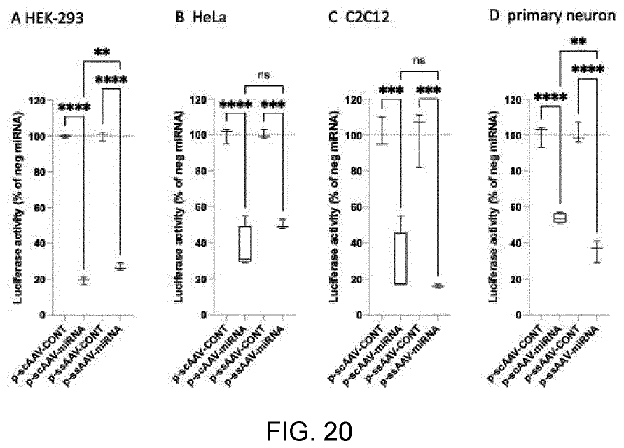

To test this issue, we cloned the vMiX system in scAAV and ssAAV ITR cassettes and compared their performance in the DLR activity assay in transfected cells (FIG. 20). Both plasmids showed 19-50% knockdown in various cell types suggesting miRNA generated from either sc or ss plasmids was efficient at knocking down a target gene.

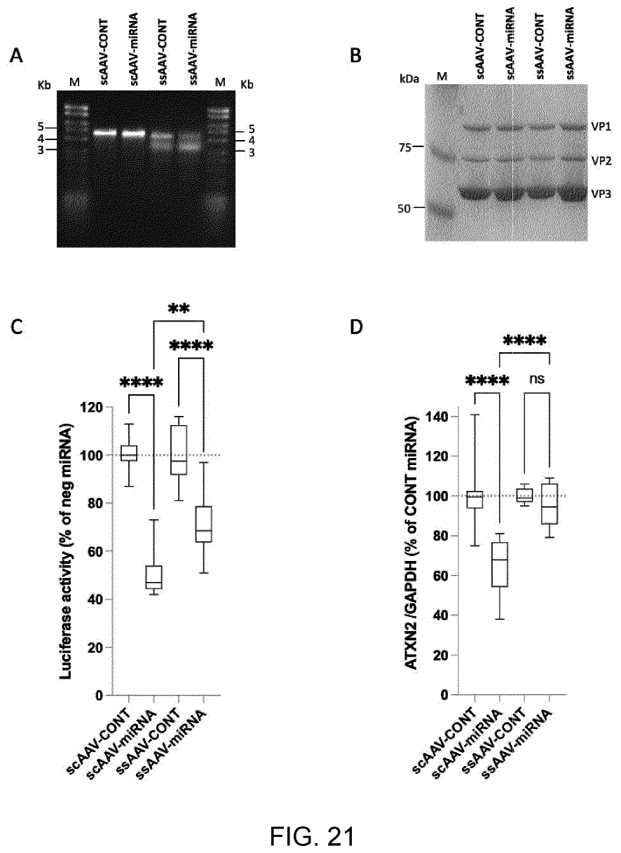

Subsequently, we generated scAAV and ssAAV vectors for vMIX-ATXN2 to compare the differences due to the miRNA hairpin. The highest quality of fully packaged AAV particles were tested for encapsidation and transgene integrity (FIG. 21). The AAVs were tested in rat primary cortical neuron for the DLR activity to show the knockdown efficiency (FIG. 21).

Moreover, the vMiX system into a self-complementary AAV cassette, which has faster onset of transcript expression than single stranded AAV cassettes and will achieves more stable miRNA expression. The benefit of the vMix construct is that the coding sequence of the EGFP can be replaced any type of the gene of interest or adding more miRNA cassette. In this respect, this system allows us to knock down efficiently either to trace the expressing cells or knocking down the toxic genes for clinical trial.

All publications mentioned in the above specification are herein incorporated by reference. Various modifications and variations of the described methods, uses and products of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A hybrid microRNA or precursor thereof comprising:
a stem sequence from a first intronic microRNA;
a loop sequence from a second microRNA, wherein the second microRNA is different from the first intronic microRNA; and
a guide sequence that binds specifically to a target mRNA;
wherein the stem, loop, and guide sequences together form a hybrid hairpin that is processed to form a mature microRNA comprising the guide sequence, and the mature microRNA directs degradation of and/or inhibits translation of said target mRNA.

2. The hybrid microRNA or precursor thereof according to claim 1, wherein:
(i) the loop sequence is from miR-128-2;
(ii) the stem sequence is from miR-423, miR-26b, miR-126, miR-106b, miR-93, or miR-25;
(iii) the hybrid microRNA or precursor thereof is a pre-microRNA or pri-microRNA:
(iv) the hybrid microRNA or precursor thereof further comprises an intronic sequence 5′ to the stem and loop sequences;
(v) the hybrid microRNA or precursor thereof further comprises an intronic sequence 3′ to the stem and loop sequences;
(vi) the hybrid microRNA or precursor thereof comprises one or more serine/arginine rich protein (SR protein) binding sites;
(vii) the quide sequence is between 12-30 nucleotides in length;
(viii) the target mRNA is an mRNA from (a) a gene for which a mutation results in a toxic gain-of-function or (b) a gene for which suppression alleviates a pathology:
(ix) the hybrid microRNA targets the 3′ untranslated region (UTR), 5′UTR, or coding sequence (CDS) of the target mRNA;
(x) the stem and/or loop sequences are derived from a microRNA that is expressed at least in the brain; or
(xi) the loop sequence is derived from an intronic microRNA.

3. (canceled)

4. The hybrid microRNA or precursor thereof according to claim 2, wherein:
(i) the stem sequence is from miR-423, optionally wherein the stem sequence comprises the sequence of any one of SEQ ID NOs: 19-20 or a sequence having at least 85%, 90%, 95%, or 99% sequence identity to any one of SEQ ID NOs: 19-20;
(ii) the 5′ intronic sequence comprises at least 30, 50, 100, 150, or 200 nucleotide residues, optionally wherein the 5′ intronic sequence comprises 50 to 250 or 100 to 250 nucleotide residues, optionally wherein the 5′ intronic sequence comprises at least 50, 100, 150, or 200 nucleotide residues of, or the full length of SEQ ID NO: 18 or any one of SEQ ID NOs: 84-88;
(iii) the one or more SR protein binding sites are 5′ to the stem and loop sequences and comprises the sequence of: SRSF5-(SEQ ID NOs: 22 or 23), SRSF1-(SEQ ID NOs: 24 or 25), SRSF1-HMG (SEQ ID NO: 26), SRSF-(SEQ ID NO: 27), SRSF1-(SEQ ID NO: 36), SRSF6-(SEQ D NO: 37), or SRSF5-(SEQ ID NO: 38);
(iv) the SR binding site is 3′ to the stem and loop sequences and comprises the sequence of: SRSF6-(SEQ ID NO: 28), SRSF5-(SEQ ID NOs: 29 or 30), SRSF1-HMG-(SEQ ID NOs: 31 to 33), SRSF1-(SEQ ID NO: 34), SRSF2-(SEQ ID NO: 35), SRSF6-(SEQ ID NO: 39), or SRSF2-(SEQ ID NOs: 40 and 41);
(v) the loop sequence comprises the sequence of SEQ ID NO: 2 or a sequence having at least 85%, 90%, 95%, or 99% sequence identity to SEQ ID NO: 2;
(vi) the stem sequence comprises the sequence of any one of SEQ ID NOs: 19-20 and 58-67 or a sequence having at least 85%, 90%, 95%, or 99% sequence identity to any one of SEQ ID NOs: 19-20 and 58-67;
(vii) the hybrid microRNA targets the CDS of the target mRNA;
(viii) the stem and/or loop sequences are derived from a microRNA that is expressed in neurons;
(ix) the 5′ intronic sequence is derived from miR-423, miR-26b, miR-126, miR-106b, miR-93, or miR-25, optionally wherein the 5′ intronic sequence comprises the sequence of SEQ ID NO: 18 or any one of SEQ ID NOs: 84-88, or a sequence having at least 85%, 90%, 95%, or 99% sequence identity to SEQ ID NO: 18 or any one of SEQ ID NOs: 84-88;
(x) the 3′ intronic sequence is derived from miR-423, miR-26b, miR-126, miR-106b, miR-93, or miR-25, optionally wherein the 3′ intronic sequence comprises the sequence of SEQ ID NO: 21 or any one of SEQ ID NOs: 89-93, or a sequence having at least 85%, 90%, 95%, or 99% sequence identity to SEQ ID NO: 21 or any one of SEQ ID NOs: 89-93; or
(xi) the target mRNA is an mRNA from a gene selected from the group consisting of: progranulin (PGRN), Huntingtin (HTT), Ataxin 2 (ATXN2), Superoxidase dismutase 1 (SOD1), Chromosome 9 open reading frame 72 (C9orf72), Fused in Sarcoma (FUS), microtubule-associated protein tau (MAPT), Leucine-rich repeat kinase 2 (LRKK2), or alpha-synuclein (SCNA), optionally wherein the mRNA is from PGRN.

5-16. (canceled)

17. A polynucleotide encoding one or more of the hybrid microRNAs or precursors thereof according to claim 1, optionally wherein the polynucleotide is a DNA.

18. A polynucleotide according to claim 17, wherein:
(i) the polynucleotide encodes two or more hybrid microRNAs or precursors thereof, optionally wherein the hybrid microRNAs or precursors thereof comprise different stem and/or loop sequences; or
(ii) the polynucleotide encodes multiple microRNAs or precursors thereof, optionally wherein the hybrid microRNAs or precursors thereof comprise different guide sequences.

19. (canceled)

20. A polynucleotide encoding a hybrid microRNA or precursor thereof having at least 85%, 90%, 95%, or 99% sequence identity to SEQ ID NO: 115.

21. An expression cassette comprising the polynucleotide according to claim 17, further comprising a promoter sequence and/or a pre-mRNA 3′end cleavage site and/or a polyadenylation Poly (A) signal, optionally wherein the Poly (A) signal comprises the sequence of SEQ ID NOs: 55-57 or 76-83.

22. The expression cassette according to claim 21, wherein:
(i) the polynucleotide sequence encoding the hybrid microRNA or precursor thereof is located 3′ to the pre-mRNA 3′ end cleavage site and/or Poly (A) signal; or
(ii) the polynucleotide sequence encoding the hybrid hairpin is positioned between 40 to 1500 residues from the pre-mRNA 3′ end cleavage site and/or Poly (A) signal.

23. (canceled)

24. A vector comprising the polynucleotide according to claim 17.

25. The vector according to claim 24, wherein the vector is a lentivirus or adeno-associated virus (AAV), optionally wherein the vector is a self-complementary (sc) and/or single-stranded (ss) AAV.

26. A pharmaceutical composition comprising the hybrid microRNA or precursor thereof according to claim 1, wherein said pharmaceutical composition further comprises one or more pharmaceutically acceptable excipients, diluents, or carriers.

27. The pharmaceutical composition according to claim 26, further comprising a liposome.

28. A method of degrading and/or inhibiting translation of mRNA in a cell, said method comprising contacting the cell with the hybrid microRNA or precursor thereof according to claim 1.

29. A method of:
(i) reducing or inhibiting the expression of a target polynucleotide in a subject in need thereof;
(ii) treating or preventing a disease, disorder, or condition in a subject in need thereof. optionally wherein the disease, disorder, or condition is a neurodegenerative disease; or
(iii) treating or preventing a neurodegenerative disease in a subject in need thereof. the method comprising administering to the subject a therapeutically effective amount of the hybrid microRNA or precursor thereof according to claim 1.

30. (canceled)

31. The method according to claim 29, wherein the neurodegenerative disease is selected from the group consisting of: Huntington's disease (HD), Parkinson's disease (PD), Fronto-temporal dementia (FTD), Alzheimer's disease (AD), Amyotrophic Lateral Sclerosis (ALS), Creutzfeldt-Jakob disease (CJD), Spinocerebellar Ataxia 2 (SCA2), and progressive supranuclear palsy (PSP).

32. (canceled)

33. A method of producing a hybrid microRNA or precursor thereof that suppresses expression of a target mRNA, the method comprising:
(i) selecting, preparing, or obtaining a hybrid microRNA backbone sequence, or a polynucleotide, expression cassette, or vector that encodes said hybrid microRNA backbone sequence;
wherein the hybrid microRNA backbone sequence comprises a stem sequence from a first intronic microRNA, and a loop sequence from a second microRNA; and
(ii) cloning a guide sequence that binds specifically to a target mRNA into said hybrid microRNA backbone sequence, or into said polynucleotide, expression cassette, or vector;
wherein the stem, loop, and guide sequences together form a hybrid hairpin that is processed to form a mature microRNA comprising the guide sequence, and the mature microRNA directs degradation of and/or inhibits translation of said target mRNA.

34-35. (canceled)

36. The method according to claim 33, wherein:
(i) the guide sequence comprises 12-30 nucleotide residues;
(ii) the stem and/or loop sequences are derived from a microRNA that is expressed at least in the brain, optionally wherein the microRNA is expressed in neurons; or
(iii) the loop sequence is derived from an intronic microRNA.

37-38. (canceled)

39. The expression cassette according to claim 22, wherein:
(i) the 5′ residue of the stem sequence is positioned between 40 to 1500 residues from the pre-mRNA 3′ end cleavage site and/or Poly (A) signal;
(ii) the polynucleotide sequence encoding the hybrid hairpin is positioned between 40 to 1500 residues from the pre-mRNA 3′ end cleavage site and/or from the 3′ residue of the Poly (A) signal; or
(iii) the polynucleotide sequence encoding the hybrid hairpin is positioned between 50 to 1000, 100 to 500, or 200 to 300 residues from the pre-mRNA 3′ end cleavage site and/or Poly (A) signal.
